(19) United States
(12) Patent Application Publication
(10) Pub. No.: US 2024/0216260 A1
(43) Pub. Date: Jul. 4, 2024

(54) HAIR TREATMENT COMPOSITIONS THAT IMPART SHINE AND LUSTER

(71) Applicant: L'OREAL, Paris (FR)

(72) Inventors: Kazumitsu KAWAKAMI, Westfield, NJ (US); Liliana XAVIER, Elizabeth, NJ (US); Kayla STENCEL, Hoboken, NJ (US)

(21) Appl. No.: 18/067,076

(22) Filed: Dec. 16, 2022

(51) Int. Cl. A61K 8/894; A61K 8/06; A61K 8/34; A61K 8/44; A61Q 5/12

(52) U.S. Cl. CPC A61K 8/894; A61K 8/062; A61K 8/342; A61K 8/345; A61K 8/44; A61Q 5/12; A61K 2800/48

(57) ABSTRACT

Disclosed are hair treatment compositions particularly useful for imparting shine and luster to hair. The compositions include: (a) one or more polyether-modified polysiloxanes; (b) one or more glycols; (c) one or more cationic conditioning agents; (d) one or more fatty alcohols; and (e) water. The compositions impart shine and luster to hair indicative of natural, healthy hair, without causing build-up, stickiness, and heaviness, regardless of whether the compositions are used as a leave-in product or rinse-out product.

### FIELD OF THE DISCLOSURE

The present disclosure relates to hair treatment compositions that impart a desirable shine and luster to hair and to methods for treating hair to impart and enhance shine and luster to hair. Regardless of whether the hair treatment compositions are rinsed from the hair as a rinse-out (or rinse-off) product or allowed to remain the hair as a leave-on product, they impart a noticeable lightweight, glistening finish and to the hair.

### BACKGROUND

Hair loses its natural shine for a variety of reasons. Under low humidity conditions hair loses moisture causing it to lose shine and become brittle. Loss of shine can also result from repeated chemical treatments. Even regular cleansing of the hair tends to negatively impact its natural shine due to removal of natural oils. Shampoos include anionic surfactants having detersive qualities to cleanse and lift contamination from the hair but inevitably the process is indiscriminate and removes natural oils that that are beneficial. Loss of shine due to any number of these various situations can leave the hair damaged having a matted and dull appearance.

Application of styling products having film-forming properties have been used to coat the hair and prevent external damaging assaults while maintaining preventing natural oils, etc., from being stripped from the hair. Although such products provide temporary benefits, they tend to weigh down the hair, build-up over time, and become sticky in high humidity conditions. Consumers also complain that hair treated with these types of products can appear and feel unwashed and matted. Furthermore, products that rely on film-forming polymers and films are typically used exclusively as leave-on products. Upon washing the hair any benefit provided by the product is eliminated.

Oils, waxes, and other fatty materials have also used in products to provide shine and moisturizing properties to hair. Such products attempt to replace the natural oils removed from hair due to shampooing, chemical treatments, environmental factors, etc. with supplemental materials. Although simply replacing oils removed from hair seems logical, consumers complain the hair feels greasy and can be difficult to style.

Consumers seek products that impart shine and luster to hair indicative of natural, healthy hair, without causing build-up, stickiness, and heaviness. The hair should feel light, be free flowing, and have a natural appearance.

### SUMMARY OF THE DISCLOSURE

The present disclosure relates to hair treatment compositions and methods that impart a desirable shine and luster to hair. Regardless of whether the hair treatment compositions are rinsed from the hair as a rinse-out (or rinse-off) product or remain the hair as a leave-on product, they impart a noticeable lightweight, glistening finish and shine to the hair. The compositions are easy to use and provide long-lasting results that withstand rinsing and subsequent cleansing. Furthermore, the compositions are unique because they do not require film-forming styling polymers that form a film or layer on the hair, nor do they require hydrocarbon-based oils. The compositions impart shine and luster to hair indicative of natural, healthy hair, without causing build-up, stickiness, and heaviness. The treated hair has a lightweight feel, is free flowing, and exhibits a natural and healthy appearance.

The hair treatment compositions are typically an oil-in-water emulsion and includes:
(a) one or more polyether-modified polysiloxanes;
(b) one or more glycols selected from ethylene glycol, propylene glycol, butylene glycol, pentylene glycol, dipropylene glycol, or a combination thereof;
(c) one or more cationic conditioning agents;
(d) of one or more fatty alcohols; and
(e) water.

Nonlimiting examples of polyether-modified polysilxoanes includes PEGylated dimethicones, bis-PEGylated dimethicones, PEG/PPG dimethicones, or a combination thereof. Nonlimiting examples of PEGylated dimethicones include PEG-10 dimethicone, PEG-9 dimethicone, PEG-8 dimethicone, PEG-3 dimethicone, PEG-7 dimethicone, PEG-12 dimethicone, PEG-11 methyl ether dimethicone, PEG-32-dimethicone, PEG-14 dimethicone, PEG-17 dimethicone, or a combination thereof.

Nonlimiting examples of bis-PEGylated dimethicones include bis-PEG-[10-20] dimethicone, bis-PEG-10 dimethicone, bis-PEG-12 dimethicone, bis-PEG-17 dimethicone, bis-PEG-20 dimethicone, or a combination thereof.

Nonlimiting examples of PEG/PPG dimethicones include PEG/PPG-3/10 dimethicone, PEG/PPG-4/12 dimethicone, PEG/PPG-6/11 dimethicone, PEG/PPG-8/14 dimethicone, PEG/PPG-14/4 dimethicone, PEG/PPG-15/15 dimethicone, PEG/PPG-16/2 dimethicone, PEG/PPG-17/18 dimethicone, PEG/PPG-18/18 dimethicone, PEG/PPG-19/19 dimethicone, PEG/PPG-20/6 dimethicone, PEG/PPG-20/15 dimethicone, PEG/PPG-20/20 Dimethicone, PEG/PPG-20/23 dimethicone, PEG/PPG-20/29 dimethicone, PEG/PPG-22/23 dimethicone, PEG/PPG-22/24 dimethicone, PEG/PPG-23/6 dimethicone, PEG/PPG-25/25 dimethicone, PEG/PPG-27/27, bis-PEG/PPG 18/6 dimethicone, bis-PEG/PPG-20/20 dimethicone, PEG/PPG-18/18 dimethicone, cetyl PEG/PPG-10/1 dimethicone, lauryl PEG/PPG-18/18 methicone, PEG/PPG 20/22 butyl ether dimethicone, PEG/PPG 23/6 dimethicone, PEG/PPG 20/15 dimethicone, PEG/PPG 20/23 dimethicone, or a combination thereof.

A preferred glycol is propylene glycol.

Nonlimiting examples of cationic conditioning agents include cationic surfactants, cationic polymers, or a combination thereof. Nonlimiting examples of cationic surfactants include quaternary ammonium compounds, fatty dialkylamines, fatty amidoamines, salts thereof, or a combination thereof.

Nonlimiting examples of quaternary ammonium compounds include cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, or a combination thereof.

Nonlimiting examples of fatty dialkylamines include oleamidopropyl dimethylamine, linoleamidopropyl dimethylamine, isostearamidopropyl dimethylamine, oleyl hydroxyethyl imidazoline, stearamidopropyldimethylamine, behenamidopropyl-dimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, salts thereof, or a combination thereof.

Fatty alcohols can be saturated (alkyl) or unsaturated (alkenyl), linear or branched, optionally substituted with one or more hydroxyl groups. Nonlimiting examples of fatty alcohols include decyl alcohol, undecyl alcohol, dodecyl alcohol, myristyl alcohol, cetyl alcohol, stearyl alcohol, isostearyl alcohol, isocetyl alcohol, behenyl alcohol, linalool, oleyl alcohol, myricyl alcohol, or a combination thereof.

The hair treatment compositions optionally include one or more nonionic thickening polymers. Nonlimiting examples of nonionic thickening polymers include polysaccharides, polysaccharide derivatives, or a combination thereof. more specific but nonlimiting examples of nonionic thickening polymers include sclerotium gum, guar gums, guar gum derivatives, xanthan gum, hydroxyalkyl celluloses optionally modified with a hydrophobic group, inulins, carrageenans, carrageenan derivatives, or a combination thereof.

The hair treatment compositions optionally include one or more surfactants, which are different from the cationic conditioning agents, which can include cationic surfactants. Surfactants other than cationic surfactants can be amphoteric (zwitterionic), nonionic, or anionic. It is more common for the hair treatment compositions to include one or more amphoteric surfactants and/or nonionic surfactants than anionic surfactants. Therefore, in certain embodiments, the hair treatment compositions are free or essentially free from anionic surfactants. In a preferred embodiment, the hair treatment compositions include one or more amphoteric surfactants. Nonlimiting examples of amphoteric surfactants include alkyl betaines, alkyl sultaines, alkyl amphoacetates, alkyl amphoproprionates, or a combination thereof. Alkyl betaines are particularly useful.

Nonlimiting examples of alkyl betaines include cocobetaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, and mixtures thereof. Typically, at least one betaine compound is selected from coco betaine, cocamidopropyl betaine, behenyl betaine, capryl/capramidopropyl betaine, and lauryl betaine, and mixtures thereof. Particularly preferred betaines include coco betaine and cocamidopropyl betaine.

The hair treatment compositions optionally include one or more amino-functionalized silicone oils. Non-limiting examples of amino-functionalized silicone oils include amodimethicone, bis-hydroxy/methoxy amodimethicones, bis-cetearyl amodimethicone, amodimethicone, bis(C13-15 alkoxy) PG amodimethicones, aminopropyl phenyl trimethicones, aminopropyl dimethicones, bis-amino PEG/PPG-41/3 aminoethyl PG-propyl dimethicones, or a mixture thereof. In various embodiments, amodimethicone is particularly useful.

In various embodiments, the compositions have a translucent or opaque appearance. In further embodiments, the hair treatment compositions are in the form of a cream, gel, lotion, rinse, and the like.

The compositions are particularly useful for improving the look and feel of hair. In particular, the hair treatment compositions are useful for: (i) imparting shine and luster to hair; (ii) improving texture and feel of hair; (iii) preventing or reducing frizz, especially in high humidity conditions; and (iv) improving the appearance of hair; wherein the methods typically comprise applying a hair treatment composition disclosed herein to the hair. The hair treatment composition can be applied to the hair as a leave-on product (i.e., allowed to remain on the hair without rinsing) or as a rinse-out product (i.e., rinsed from the hair prior to subsequently styling the hair).

### DETAILED DESCRIPTION OF THE DISCLOSURE

The instant disclosure describes hair treatment compositions, methods for treating hair with the hair treatment compositions, and kits comprising the hair treatment compositions. The hair treatment compositions advantageously impart or enhance shine and luster to hair, reduce frizz, and improve the overall appearance and feel of hair. Hair treated with the compositions appears natural looking and healthy and is not greasy or weighed down. These benefits and other are described in more detail throughout the disclosure. The hair treatment compositions of the disclosure are typically oil-in-water emulsions and include:
(a) about 1 to about 10 wt. % of one or more polyether-modified polysiloxanes;
(b) about 2 to about 20 wt. % of one or more glycols selected from ethylene glycol, propylene glycol, butylene glycol, pentylene glycol, dipropylene glycol, or a combination thereof, preferably propylene glycol;
(c) about 1 to about 12 wt. % of one or more cationic conditioning agents;
(d) about 1 to about 15 wt. % of one or more fatty alcohols
(e) about 55 to about 95 wt. % of water;
   all weight percentages being based on a total weight of the composition.

In addition to the above, in various embodiments, the hair treatment compositions optionally include: (f) one or more nonionic thickening polymers; (g) one or more surfactants other than cationic surfactants of the one or more cationic conditioning agents of (c); (h) one or more amino-functionalized silicone oils; and/or (i) one or more miscellaneous ingredients.

The compositions are particularly useful for improving the look and feel of hair. In particular, the hair treatment compositions impart shine and luster to hair, improve texture and feel of hair, and prevent or reducing frizz, especially in high humidity conditions. Methods of using the hair treatment compositions typically include applying a hair treatment composition to the hair. The hair treatment composition can be applied to the hair as a leave-on product (i.e., allowed to remain on the hair without rinsing) or as a rinse-out product (i.e., rinsed from the hair prior to styling the hair).

### (a) Polyether-Modified Polysiloxanes

Nonlimiting examples of polyether-modified polysiloxanes include PEGylated dimethicones, bis-PEGylated dimethicones, PEG/PPG dimethicones, or a combination thereof. Exemplary, but nonlimiting examples of PEGylated dimethicones include PEG-10 dimethicone, PEG-9 dimethicone, PEG-8 dimethicone, PEG-3 dimethicone, PEG-7 dimethicone, PEG-12 dimethicone, PEG-14 dimethicone, PEG-17 dimethicone, or a combination thereof.

Exemplary, but nonlimiting examples, of bis-PEGylated dimethicones include bis-PEG-[10-20] dimethicone, bis-PEG-10 dimethicone, bis-PEG-12 dimethicone, bis-PEG-17 dimethicone, bis-PEG-20 dimethicone, or a combination thereof.

Exemplary, but nonlimiting examples of PEG/PPG dimethicones include PEG/PPG-3/10 dimethicone, PEG/PPG-4/12 dimethicone, PEG/PPG-6/11 dimethicone, PEG/PPG-8/14 dimethicone, PEG/PPG-14/4 dimethicone, PEG/PPG-15/15 dimethicone, PEG/PPG-16/2 dimethicone, PEG/PPG-17/18 dimethicone, PEG/PPG-18/18 dimethicone, PEG/PPG-19/19 dimethicone, PEG/PPG-20/6 dimethicone, PEG/PPG-20/15 dimethicone, PEG/PPG-20/20 Dimethicone, PEG/PPG-20/23 dimethicone, PEG/PPG-20/29 dimethicone, PEG/PPG-22/23 dimethicone, PEG/PPG-22/24 dimethicone, PEG/PPG-23/6 dimethicone, PEG/PPG-25/25 dimethicone, PEG/PPG-27/27, bis-PEG/PPG 18/6 dimethicone, or a combination thereof.

Useful polyether-modified polysiloxanes include compounds of the following formula:

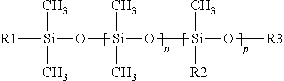

Radicals R1 and R3 independently constitute a hydrogen atom, an alkyl group with from about 1 to about 30 C-atoms, an alkoxy group with from about 1 to about 30 C-atoms or a possibly substituted phenyl group,

Radical R2 constitutes the group —C_{c}H_{2c}—O—(C₂H₄O—)ₐ(C₃H₆O—)_{b}R5,

Radical R5 constitutes a hydrogen atom or a linear or branched alkyl group with 1 to 16 C-atoms,

n is a number from about 0 to about 500,

p is a number from about 1 to about 50,

a is a number from about 0 to about 50,

b is a number from about 0 to about 50,

a+b are at least 1, and

c is a number from about 1 to about 4.

In certain embodiments, polyether-modified polysiloxanes of the general structural formula (I) are:

TABLE 1

| | R1, R3 | R2 | R5 | n | p | a | b | c |
|---|---|---|---|---|---|---|---|---|
| 1 | H, methyl | —C_{c}H_{2c}—O—(C₂H₄O)ₐ—(C₃H₆O)_{b}—R₅ | H, methyl | 1-500 | 5-50 | 15-25 | 15-25 | 3 |
| 2 | H, methyl | —C_{c}H_{2c}—O—(C₂H₄O)ₐ—(C₃H₆O)_{b}—R₅ | H, methyl | 1-500 | 5-50 | 5-20 | 0.1 | 3 |
| 3 | H, methyl | —C_{c}H_{2c}—O—(C₂H₄O)ₐ—(C₃H₆O)_{b}—R₅ | H, methyl | 0.1 | 1-3 | 5-10 | 0.1 | 2 |
| 4 | H, methyl | —C_{c}H_{2c}—O—(C₂H₄O)ₐ—(C₃H₆O)_{b}—R₅ | H, methyl | 1-500 | 5-50 | 20-30 | 20-30 | 3 |
| 5 | H, methyl | —C_{c}H_{2c}—O—(C₂H₄O)ₐ—(C₃H₆O)_{b}—R₅ | H, methyl | 1-500 | 5-50 | 10-25 | 10-25 | 3 |
| 6 | H, methyl | —C_{c}H_{2c}—O—(C₂H₄O)ₐ—(C₃H₆O)_{b}—R₅ | H, methyl | 1-500 | 2-50 | 10-30 | 2-10 | 3 |
| 7 | H, methyl | —C_{c}H_{2c}—O—(C₂H₄O)ₐ—(C₃H₆O)_{b}—R₅ | | 1-500 | 2-50 | 10-20 | 1-10 | 3 |

TABLE 2

| | R1, R3 | R2 | R5 | n | p | a | b | c |
|---|---|---|---|---|---|---|---|---|
| 1 | Methyl | —C_{c}H_{2c}—O—(C₂H₄O)ₐ—(C₃H₆O)_{b}—R₅ | H, methyl | 5-500 | 7-50 | 15-20 | 15-20 | 3 |
| 2 | Methyl | —C_{c}H_{2c}—O—(C₂H₄O)ₐ—(C₃H₆O)_{b}—R₅ | H, methyl | 5-500 | 7-50 | 7-15 | 0 | 3 |
| 3 | Methyl | —C_{c}H_{2c}—O—(C₂H₄O)ₐ—(C₃H₆O)_{b}—R₅ | H, methyl | 0.1 | 1 | 6-8 | 0 | 2 |
| 4 | Methyl | —C_{c}H_{2c}—O—(C₂H₄O)ₐ—(C₃H₆O)_{b}—R₅ | H, methyl | 5-500 | 7-50 | 20-25 | 20-25 | 3 |
| 5 | Methyl | —C_{c}H_{2c}—O—(C₂H₄O)ₐ—(C₃H₆O)_{b}—R₅ | H, methyl | 5-500 | 7-50 | 15-20 | 15-20 | 3 |
| 6 | Methyl | —C_{c}H_{2c}—O—(C₂H₄O)ₐ—(C₃H₆O)_{b}—R₅ | H, methyl | 5-500 | 2-50 | 15-25 | 2-8 | 3 |
| 7 | Methyl | —C_{c}H_{2c}—O—(C₂H₄O)ₐ—(C₃H₆O)_{b}—R₅ | H. methyl | 5-500 | 2-50 | 10-15 | 1-5 | 3 |

TABLE 3

| | R1, R3 | R2 | R5 | n | p | a | b | c |
|---|---|---|---|---|---|---|---|---|
| 1 | Methyl | —C_{c}H_{2c}—O—(C₂H₄O)ₐ—(C₃H₆O)_{b}—R₅ | Methyl | 10-500 | 10-50 | 18 | 18 | 3 |
| 2 | Methyl | —C_{c}H_{2c}—O—(C₂H₄O)ₐ—(C₃H₆O)_{b}—R₅ | Methyl | 10-500 | 10-50 | 12 | 0 | 3 |
| 3 | Methyl | —C_{c}H_{2c}—O—(C₂H₄O)ₐ—(C₃H₆O)_{b}—R₅ | Methyl | 10-500 | 10-50 | 14 | 0 | 3 |
| 4 | Methyl | —C_{c}H_{2c}—O—(C₂H₄O)ₐ—(C₃H₆O)_{b}—R₅ | Methyl | 0 | 1 | 7 | 0 | 2 |
| 5 | Methyl | —C_{c}H_{2c}—O—(C₂H₄O)ₐ—(C₃H₆O)_{b}—R₅ | Methyl | 10-500 | 10-50 | 22 | 24 | 3 |
| 6 | Methyl | —C_{c}H_{2c}—O—(C₂H₄O)ₐ—(C₃H₆O)_{b}—R₅ | Methyl | 10-500 | 10-50 | 17 | 18 | 3 |
| 7 | Methyl | —C_{c}H_{2c}—O—(C₂H₄O)ₐ—(C₃H₆O)_{b}—R₅ | Methyl | 10-500 | 5-50 | 20 | 6 | 3 |
| 8 | Methyl | —C_{c}H_{2c}—O—(C₂H₄O)ₐ—(C₃H₆O)_{b}—R₅ | Methyl | 10-500 | 5-50 | 14 | 4 | 3 |

Polyether-modified polysiloxane compounds listed in the above Tables 1-3, are commercially available under the following trade names, for example:
DOW CORNING 190 (INCI: PEG/PPG-18/18 Dimethicone),
DOW CORNING 193 (INCI: PEG-12 Dimethicone),
ABIL® B 8843 (INCI: PEG-14 Dimethicone),
SILWET L-77 (INCI: Siloxane Polyalkyleneoxide Copolymer),
MIRASIL DCMO (INCI: Cyclomethicone, PEG/PPG-22/24 Dimethicone),
DOW CORNING Q2-5220 (INCI: PEG/PPG-17/18 Dimethicone),
ABIL® B 88184 (INCI: PEG/PPG-20/6 Dimethicone), and
ABIL® B 8851 (INCI: PEG/PPG-14/4 Dimethicone).

In various embodiments, the one or more polyether-modified polysiloxanes include one or more PEGylated dimethicones selected from PEG/PPG-18/18 dimethicone, PEG-12 dimethicone, PEG-14 dimethicone, siloxane polyalkyleneoxide copolymer, PEG/PPG-22/24 dimethicone, PEG/PPG-17/18 dimethicone, PEG/PPG-20/6 dimethicone, PEG/PPG-14/4 dimethicone, or mixtures thereof, preferably chosen from PEG-12 dimethicone, PEG-14 dimethicone, or a mixture thereof.

In various embodiments, the one or more polyether-modified polysiloxanes include one or more PEG/PPG dimethicones selected from PEG/PPG-3/10 dimethicone, PEG/PPG-4/12 dimethicone, PEG/PPG-6/11 dimethicone, PEG/PPG-8/14 dimethicone, PEG/PPG-14/4 dimethicone, PEG/PPG-15/15 dimethicone, PEG/PPG-16/2 dimethicone, PEG/PPG-17/18 dimethicone, PEG/PPG-18/18 dimethicone, PEG/PPG-19/19 dimethicone, PEG/PPG-20/6 dimethicone, PEG/PPG-20/15 dimethicone, PEG/PPG-20/20 Dimethicone, PEG/PPG-20/23 dimethicone, PEG/PPG-20/29 dimethicone, PEG/PPG-22/23 dimethicone, PEG/PPG-22/24 dimethicone, PEG/PPG-23/6 dimethicone, PEG/PPG-25/25 dimethicone, PEG/PPG-27/27, bis-PEG/PPG 18/6 dimethicone, or a combination thereof, preferably PEG/PPG-3/10 dimethicone, PEG/PPG-4/12 dimethicone, PEG/PPG-5/12 dimethicone, of a combination thereof.

The total amount of the one or more polyether-modified polysiloxanes in the hair treatment compositions will vary. Nonetheless, in various embodiment, the hair treatment composition includes about 1 to about 10 wt. % of the polyether-modified polysiloxanes based on the total weight of the composition. In further embodiments, hair treatment composition includes about 1 to about 8 wt. %, about 1 to about 6 wt. %, about 1 to about 5 wt. %, about 1 to about 3 wt. %, about 1.2 to about 10 wt. %, about 1.2 to about 8 wt. %, about 1.2 to about 6 wt. %, about 1.2 to about 5 wt. %, about 1.2 to about 4 wt. %, about 1.2 to about 3 wt. % of the one or more polyether-modified polysiloxanes, based on the total weight of the composition.

### (b) Glycols

Glycols are a class of compounds having two hydroxyl (—OH) groups are attached to different carbon atoms, wherein the number of carbon atoms is 2 to 8 and can be linear or branched, preferably 2 to 6 carbon atoms. The glycols typical consist of carbon and oxygen atoms (i.e., they do not include nor substituted with other compounds). Nonlimiting examples of useful glycols include propylene glycol, butylene glycol, pentylene glycol, dipropylene glycol, or a combination thereof.

The total amount of the one or more glycols in the hair treatment compositions will vary. Nonetheless, in various embodiments, the hair treatment composition includes about 2 to about 20 wt. % of the one or more glycols, based on a total weight of the composition. In further embodiments, the hair treatment composition include about 2 to about 18 wt. %, about 2 to about 15 wt. %, about 2 to about 12 wt. %, about 2 to about 10 wt. %, about 4 to about 20 wt. %, about 4 to about 18 wt. %, about 4 to about 15 wt. %, about 4 to about 12 wt. %, about 4 to about 10 wt. %, about 5 to about 20 wt. %, about 5 to about 18 wt. %, about 5 to about 15 wt. %, about 5 to about 12 wt. %, or about 5 to about 10 wt. %, based on a total weight of the hair treatment composition.

### (c) Cationic Conditioning Agents

Cationic conditioning agents are compounds having a cationic charge and compounds that are cationizable compounds and include, for example, cationic or cationizable surfactants and cationic or cationizable polymers. As used herein, the term “cationic surfactant” includes cationic surfactants and cationizable surfactants, unless expressly specified otherwise. Similarly, the term “cationic conditioning agent” includes cationic conditioning agents and cationizable conditioning agents, unless expressly specified otherwise.

### (i) Cationic Surfactants

The term “cationic surfactant” as defined by the instant disclosure as a surfactant that may be positively charged when it is contained in the hair coloring compositions. The cationic surfactant may bear one or more positive permanent charges or may contain one or more functional groups that are cationizable in the composition according to the disclosure.

Non-limiting examples of cationic surfactants include cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, and mixtures thereof, di-C12-15 alkyl dimonium chloride, C12-18 dialkyldimonium chloride, di-C12-18 alkyl dimonium chloride, dicapryl/dicaprylyl dimonium chloride, didecyldimonium chloride, dicetyldimonium chloride, ditallowdimonium chloride, dicocodimonium chloride, distearyldimonium chloride, ceteardimonium chloride, hydroxyethyl oleyl dimonium chloride, panthenyl hydroxypropyl steardimonium chloride, hydroxypropyl bis-hydroxyethyldimonium chloride, hydroxypropyl bisstearyldimonium chloride, hydroxycetyl hydroxyethyl dimonium chloride, hydroxyethyl behenamidopropyl dimonium chloride, behenamidopropyl PG-dimonium chloride, cocamidopropyl PG-dimonium chloride, oleamidopropyl PG-dimonium chloride, behentrimonium chloride, and mixtures thereof.

In some embodiments, the cationic surfactant is selected from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, and mixtures thereof.

In some embodiments, the cationic surfactant is cetrimonium chloride, behentrimonium chloride, or a mixture thereof.

In some embodiments, the cationic surfactant is behentrimonium chloride.

In some embodiments, the cationic surfactant is cetrimonium chloride.

In some embodiments, the cationic surfactant is stearamidopropyl dimethylamine.

Additional nonlimiting examples of cationic surfactants include behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The cationic surfactant(s) may also be chosen from optionally polyoxyalkylenated, primary, secondary or tertiary fatty amines, or salts thereof, and quaternary ammonium salts, and mixtures thereof.

In some cases, it is useful to use salts such as chloride salts of the quaternary ammonium compounds.

The fatty amines generally comprise at least one C8-C30 hydrocarbon-based chain.

Examples of quaternary ammonium salts that may especially be mentioned include: those corresponding to the general formula below:

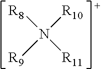

in which the groups R₈ to R₁₁, which may be identical or different, represent a linear or branched, saturated or unsaturated aliphatic group comprising from 1 to 30 carbon atoms, or an aromatic group such as aryl or alkylaryl, at least one of the groups R₈ to R₁₁ denoting a group comprising from 8 to 30 carbon atoms and, in some embodiments, from 12 to 24 carbon atoms. The aliphatic groups may comprise heteroatoms especially such as oxygen, nitrogen, sulfur and halogens. The aliphatic groups are chosen, for example, from C₁-C₃₀ alkyl, C₂-C₃₀ alkenyal, C₁-C₃₀ alkoxy, polyoxy(C₂-C₆)alkylene, C₁-C₃₀ alkylamide, (C₁₂-C₂₂)alkylamido(C₂-C₆)alkyl, (C₁₂-C₂₂)alkyl acetate and C₁-C₃₀ hydroxyalkyl groups; X⁻ is an anion chosen from the group of halides, phosphates, acetates, lactates, (C₁-C₄)alkyl sulfates, and (C₁-C₄)alkyl- or (C₁-C₄)alkylarylsulfonates.

Among the quaternary ammonium salts of formula (III), those that are preferred are, on the one hand, tetraalkylammonium salts, for instance dialkyldimethylammonium or alkyltrimethylammonium salts in which the alkyl group contains approximately from 12 to 22 carbon atoms, in particular behenyltrimethylammonium, distearyldimethylammonium, cetyltrimethylammonium or benzyldimethylstearylammonium salts, or, on the other hand, oleocetyldimethylhydroxyethylammonium salts, palmitylamidopropyltrimethylammonium salts, stearamidopropyltrimethylammonium salts and stearamidopropyldimethylcetearylammonium salts.

Also useful are quaternary ammonium salts of imidazoline, such as, for example, those of formula below:

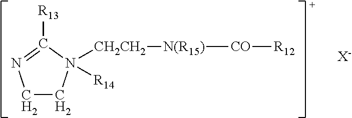

in which R₁₂ represents an alkenyl or alkyl group comprising from 8 to 30 carbon atoms, derived for example from tallow fatty acids, R₁₃ represents a hydrogen atom, a C₁-C₄ alkyl group or an alkyl or alkenyl group comprising from 8 to 30 carbon atoms, R₁₄ represents a C₁-C₄ alkyl group, R₁₅ represents a hydrogen atom or a C₁-C₄ alkyl group, X⁻ is an anion chosen from the group of halides, phosphates, acetates, lactates, alkyl sulfates, alkyl- or alkylaryl-sulfonates in which the alkyl and aryl groups, in some embodiments, comprise, respectively, from 1 to 20 carbon atoms and from 6 to 30 carbon atoms. R₁₂ and R₁₃, in some embodiments, denote a mixture of alkenyl or alkyl groups containing from 12 to 21 carbon atoms, derived for example from tallow fatty acids, R₁₄, in some embodiments, denotes a methyl group, and R₁₅, in some embodiments, denotes a hydrogen atom. Such a product is sold, for example, under the name REWOQUAT W 75 by the company Rewo.

Useful quaternary diammonium or triammonium salts includes those of the formula:

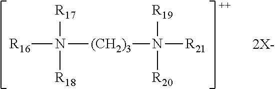

in which R₁₆ denotes an alkyl radical comprising approximately from 16 to 30 carbon atoms, which is optionally hydroxylated and/or interrupted with one or more oxygen atoms, R₁₇ is chosen from hydrogen or an alkyl radical comprising from 1 to 4 carbon atoms or a group (R₁₆ₐ)(R₁₇ₐ)(R₁₈ₐ)N—(CH₂)₃, R₁₆ₐ, R₁₇ₐ, R₁₈ₐ, R₁₈, R₁₉, R₂₀ and R₂₁, which may be identical or different, being chosen from hydrogen and an alkyl radical comprising from 1 to 4 carbon atoms, and X⁻ is an anion chosen from the group of halides, acetates, phosphates, nitrates and methyl sulfates. Such compounds are, for example, Finquat CT-P, sold by the company Finetex (Quaternium 89), and Finquat CT, sold by the company Finetex (Quaternium 75).

Useful cationic/cationizable surfactants, including cationizable surfactants together with an acid neutralizer, include those of the general structure R4-A-R5-B wherein R4 is a saturated or unsaturated, straight or branched alkyl chain with 8 to 24 C atoms, R5 is a straight or branched alkyl chain with 1 to 4 C atoms, A is selected from:

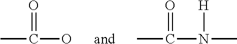

and B is selected from

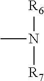

wherein R₆ and R₇ are the same or different is H or an alkyl chain with 1 to 4 C atoms, hydroxyl alkyl chain with 1 to 4 C atoms and di hydroxyl alkyl chain with 2 to 4 C atoms, and

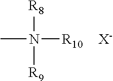

wherein R₈ and R₉ are the same or different, an alkyl chain with 1 to 4 C atoms, hydroxyl alkyl chain with 1 to 4 C atoms and di hydroxyl alkyl chain with 2 to 4 C atoms, R.sub.10 is an alkyl chain with 1 to 4 C atoms, hydroxyl alkyl chain with 1 to 4 C atoms or di hydroxyl alkyl chain with 2 to 4 C atoms.

In some instances, R₄ is saturated or unsaturated, straight or branched alkyl chain with 10 to 24C atoms, in some embodiments, 12 to 22 C atoms and R₅ is straight or branched alkyl group with 1 to 4 C atoms, and A, B, R₆ to R₁₀ are same as above.

Non-limiting suitable examples are stearyloxypropyl amine, palmityloxypropyl amine, stearyloxypropyldimethyl amine, stearyloxypropyldiethyl amine, stearyloxyethylyldimethyl amine, stearyloxyethyl amine, myristyloxypropyl amine, myristyloxypropyldimethyl amine, palmitamidopropyl amine, palmitamidopropyl methylamine, palmitamidopropyl diethylamine, palmitamidopropyl dibutylamine, palmitamidopropyl buylamine, palmitamidopropyl dipropylamine, palmitamidopropyl propylamine, palmitamidopropyl dihydroxyethylamine, palmitamidopropyl hydroxyethylamine, palmitamidopropyl dihydroxypropylamine, palmitamidopropyl hydroxypropylamine, lauramidopropyl amine, lauramidopropyl methylamine, lauramidopropyl diethylamine, lauramidopropyl dibutylamine, lauramidopropyl buylamine, lauramidopropyl dipropylamine, lauramidopropyl propylamine, lauramidopropyl dihydroxyethylamine, lauramidopropyl hydroxyethylamine, lauramidopropyl dihydroxypropylamine, lauramidopropyl hydroxypropylamine, stearamidopropyl amine, stearamidopropyl dimethylamine, steara midopropyl diethylamine, stearamidopropyldibutylamine, stearamidopropyl butylamine, stearamidopropyl dipropylamine, behenamidopropyl propylamine, behenamidopropyl dihydroxyethylamine, behenamidopropyl hydroxyethylamine, behenamidopropyl dihydroxypropylamine, behenamidopropyl hydroxypropylamine, behenamidopropyl amine, behenamidopropyl methylamine, behenamidopropyl diethylamine, behenamidopropyl dibutylamine, behenamidopropyl butylamine, behenamidopropyl dipropylamine, behenamidopropyl propylamine, behenamidopropyl dihydroxyethylamine, behenamidopropyl hydroxyethylamine, behenamidopropyl dihydroxypropylamine, behenamidopropyl hydroxypropylamine, dipalmitamidopropyl methylamine, dipalmitamidopropyl ethylamine, dipalmitamidopropyl butylamine, dipalmitamidopropyl propylamine, dipalmitamidopropyl hydroxyethylamine, dipalmitamidopropyl hydroxypropylamine, dilauramidopropyl amine, dilauramidopropyl methylamine, dilauramidopropyl buylamine, dilauramidopropyl hydroxyethylamine, dilauramidopropyl hydroxypropylamine, distearamidopropyl amine, distearamidopropyl methylamine, dibehenamidopropyl propylamine, dibehenamidopropyl hydroxyethylamine, palmitoamidopropyl trimethyl ammonium chloride, stearamidopropyl trimethylammonium chloride, behenamidopropyl tri hydroxyethalmonium chloride, distearylamidopropyl dimethyl ammonium chloride, dicetylamidodihydroxyethyl ammonium chloride, palmitoylpropyl amine, palmitoylpropyl methylamine, palmitoylpropyl diethylamine, palmitoylpropyl dibutylamine, palmitoylpropyl buylamine, palmitoylpropyl dipropylamine, palmitoylpropyl propylamine, palmitoylpropyl dihydroxyethylamine, palmitoylpropyl hydroxyethylamine, palmitoylpropyl dihydroxypropylamine, palmitoylpropyl hydroxypropylamine, myristoylpropyl amine, myristoylpropyl methylamine, myristoylpropyl diethylamine, myristoylpropyl dibutylamine, myristoylpropyl buylamine, myristoylpropyl dipropylamine, myristoylpropyl propylamine, myristoylpropyl dihydroxyethylamine, myristoylpropyl hydroxyethylamine, myristoylpropyl dihydroxypropylamine, myristoylpropyl hydroxypropylamine, stearoylpropyl amine, stearoylpropyl methylamine, stearoylpropyl diethylamine, stearoylpropyl dibutylamine, stearoylpropyl butylamine, stearoylpropyl dipropylamine, behenylpropyl propylamine, behenylpropyl dihydroxyethylamine, behenylpropyl hydroxyethylamine, behenylpropyl dihydroxypropylamine, behenylpropyl hydroxypropylamine, behenylpropyl amine, behenylpropyl methylamine, behenylpropyl diethylamine, behenylpropyl dibutylamine, behenylpropyl butylamine, behenylpropyl dipropylamine, behenylpropyl propylamine, behenylpropyl dihydroxyethylamine, behenylpropyl hydroxyethylamine, behenylpropyl dihydroxypropylamine, behenylpropyl hydroxypropylamine, dipalmitoylpropyl methylamine, dipalmitoylpropyl ethylamine, dipalmitylpropyl butylamine, dipalmitylpropyl propylamine, dipalmitylpropyl hydroxyethylamine, dipalmitylpropyl hydroxypropylamine, dilauroylpropyl amine, dilauroylpropyl methylamine, dilauroylpropyl buylamine, dilauroylpropyl hydroxyethylamine, dilauroylpropyl hydroxypropylamine, distearylpropyl amine, distearylpropyl methylamine, dibehenylpropyl propylamine, dibehenylpropyl hydroxyethylamine, palmitylpropyl trimethyl ammonium chloride, stearylpropyl trimethylammonium chloride, behenylpropyl tri hydroxyethalmonium chloride, distearylpropyl dimethyl ammonium chloride, dicetyldihydroxyethyl ammonium chloride, dioleoylethylhydroxyethylmonium methosulfate, and dicocoylethylhydroxyethylmonium methosulfate.

Cationizable surfactants may be chosen from fatty alkylamines and fatty dialkylamines. In some cases, the fatty dialkylamines may be fatty dimethylamines. Non-limiting examples include dimethyl lauramine, dimethyl behenamine, dimethyl cocamine, dimethyl myristamine, dimethyl palmitamine, dimethyl stearamine, dimethyl tallowamine, dimethyl soyamine, and mixtures thereof.

Fatty dialkylamines include fatty amidoamine compounds, their salts, and mixtures thereof. Non-limiting examples include oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, and palmitamidopropyl dimethylamine.

In preferred embodiments, at least one of the one or more cationic surfactants is a dialkyl dimonium compound, preferably a dialkyl dimonium halide compound. Non-limiting examples of dialkyl dimonium compounds include di-C12-15 alkyl dimonium chloride, C12-18 dialkyldimonium chloride, di-C12-18 alkyl dimonium chloride, dicapryl/dicaprylyl dimonium chloride, didecyldimonium chloride, dicetyldimonium chloride, ditallowdimonium chloride, dicocodimonium chloride, distearyldimonium chloride, ceteardimonium chloride, hydroxyethyl oleyl dimonium chloride, panthenyl hydroxypropyl steardimonium chloride, hydroxypropyl bis-hydroxyethyldimonium chloride, hydroxypropyl bisstearyldimonium chloride, hydroxycetyl hydroxyethyl dimonium chloride, hydroxyethyl behenamidopropyl dimonium chloride, behenamidopropyl PG-dimonium chloride, cocamidopropyl PG-dimonium chloride, oleamidopropyl PG-dimonium chloride, behenamidopropyl ethyldimonium ethosulfate, cocoamidopropyl ethyldimonium ethosulfate, dicetyldimonium chloride hydroxyethyl behenamidopropyl dimonium chloride, PEG-2-cocomonium chloride, PPG-9 diethylmonium chloride, PPG-25 diethylmonium chloride, PPG-40 diethylmonium chloride, stearamidopropyl ethyl dimonium ethosulfate, steardimonium hydroxypropyl hydrolyzed wheat protein, steardimonium hydroxypropyl hydrolyzed collagen, wheat germamidopropalkonium chloride, wheat germamidopropyl ethyldimonium ethosulfate, and mixtures thereof.

Preferably, the one or more cationic surfactants are selected from quaternary ammonium compounds, fatty dialkylamines, or a combination thereof, preferably wherein:
the one or more quaternary ammonium compounds are selected from cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, or a combination thereof, preferably behentrimonium chloride; and
the one or more fatty dialkylamines are selected from oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, salts thereof, or a combination thereof, preferably stearamidopropyl dimethylamine.

The total amount of the one or more cationic surfactants in the hair treatment compositions will vary. Nonetheless, in various embodiments, the hair treatment composition includes about 0.5 to about 10 wt. % of the one or more cationic surfactants, based on the total weight of the hair treatment composition. In further embodiments, the hair treatment composition includes about 0.5 to about 8 wt. %, about 0.5 to about 6 wt. %, about 0.5 to about 5 wt. %, about 0.5 to about 4 wt. %, about 1 to about 10 wt. %, about 1 to about 8 wt. %, about 1 to about 6 wt. %, about 1 to about 5 wt. %, about 1 to about 4 wt. %, about 2 to about 10 wt. %, about 2 to about 8 wt. %, about 2 to about 6 wt. %, about 2 to about 5 wt. %, or about 2 to about 4 wt. %, based on a total weight of the hair treatment composition.

### (ii) Cationic Conditioning Polymers

The hair treatment composition may optionally include one or more cationic conditioning polymers (or simply “cationic polymers”) or cationizable conditioning polymers (or simply “cationizable polymers”).

Non-limiting examples of cationic polymers include copolymers of 1-vinyl-2-pyrrolidine and 1-vinyl-3-methyl-imidazolium salt (e.g., chloride salt) (referred to as Polyquaternium-16); copolymers of 1-vinyl-2-pyrrolidine and dimethylaminoethyl methacrylate (referred to as Polyquaternium-11); cationic diallyl quaternary ammonium-containing polymer including, for example, dimethyldiallyammonium chloride homopolymer and copolymers of acrylamide and dimethyldiallyammonium chloride (referred to as Polyquaternium-6 and Polyquaternium-7); polysaccharide polymers, such as cationic cellulose derivatives and cationic starch derivatives. Cationic cellulose is available as salts of hydroxyethyl cellulose reacted with trimethyl ammonium substituted epoxide (referred to as Polyquaternium-10). Another type of cationic cellulose includes the polymeric quaternary ammonium salts of hydroxyethyl cellulose reacted with lauryl dimethyl ammonium-substituted epoxide (referred to as Polyquaternium-24). Additionally, or alternatively, the cationic conditioning polymers may include or be chosen from cationic guar gum derivatives, such as guar hydroxypropyltrimonium chloride.

In certain embodiments, the one or more cationic conditioning polymers include cationic polysaccharide polymers, such as cationic cellulose, cationic starch, and cationic guar gum. In the context of the instant disclosure cationic polysaccharide polymers include cationic polysaccharides and polysaccharide derivatives (e.g., derivatized to be cationic), for example, resulting in cationic cellulose (cellulose derivatized to be cationic), cationic starch (derivatized to be cationic), cationic guar (guar derivatized to be cationic).

In various embodiments, the hair treatment compositions is free or essentially free from cationic guar and cationic guar derivatives, for example, hydroxypropyl guar hydroxypropyltrimonium chloride.

Non-limiting examples of cationic celluloses include hydroxyethylcellulose (also known as HEC), hydroxymethylcellulose, methylhydroxyethylcellulose, hydroxypropylcellulose (also known as HPC), hydroxybutylcellulose, hydroxyethylmethylcellulose (also known as methyl hydroxyethylcellulose) and hydroxypropylmethylcellulose (also known as HPMC), cetyl hydroxyethylcellulose, polyquaternium-10, polyquaternium-24, and mixtures thereof, preferably polyquaternium-10, polyquaternium-24, and mixtures thereof.

Non-limiting examples of cationic guar include guar hydroxypropyltrimonium chloride, hydroxypropyl guar hydroxypropyltrimonium chloride,

Non-limiting examples of cationic starch include starch hHydroxypropyltrimonium chloride, hydroxypropyl oxidized starch PG trimonium chloride, and a mixture thereof.

In various embodiments, the one or more cationic conditioning polymers are chosen from polyquaterniums. Nonlimiting examples include Polyquaternium-1 (ethanol, 2,2′,2″-nitrilotris-, polymer with 1,4-dichloro-2-butene and N, N,N′,N′-tetramethyl-2-butene-1,4-diamine), Polyquaternium-2, (poly[bis(2-chloroethyl) ether-alt-1,3-bis[3-(dimethylamino)propyl]urea]), Polyquaternium-4, (hydroxyethyl cellulose dimethyl diallylammonium chloride copolymer; Diallyldimethylammonium chloride-hydroxyethyl cellulose copolymer), Polyquaternium-5 (copolymer of acrylamide and quaternized dimethylammoniumethyl methacrylate), Polyquaternium-6 (poly(diallyldimethylammonium chloride)), Polyquaternium-7 (copolymer of acrylamide and diallyldimethylammonium chloride), Polyquaternium-8 (copolymer of methyl and stearyl dimethylaminoethyl ester of methacrylic acid, quaternized with dimethylsulphate), Polyquaternium-9 (homopolymer of N,N-(dimethylamino)ethyl ester of methacrylic acid, quaternized with bromomethane), Polyquaternium-10 (quaternized hydroxyethyl cellulose), Polyquaternium-11 (copolymer of vinylpyrrolidone and quaternized dimethylaminoethyl methacrylate), Polyquaternium-12 (ethyl methacrylate/abietyl methacrylate/diethylaminoethyl methacrylate copolymer quaternized with dimethyl sulfate), Polyquaternium-13 (ethyl methacrylate/oleyl methacrylate/diethylaminoethyl methacrylate copolymer quaternized with dimethyl sulfate), Polyquaternium-14 (trimethylaminoethylmethacrylate homopolymer), Polyquaternium-15 (acrylamide-dimethylaminoethyl methacrylate methyl chloride copolymer), Polyquaternium-16 (copolymer of vinylpyrrolidone and quaternized vinylimidazole), Polyquaternium-17 (adipic acid, dimethylaminopropylamine and dichloroethylether copolymer), Polyquaternium-18 (azelanic acid, dimethylaminopropylamine and dichloroethylether copolymer), Polyquaternium-19 (copolymer of polyvinyl alcohol and 2,3-epoxypropylamine), Polyquaternium-20 (copolymer of polyvinyl octadecyl ether and 2,3-epoxypropylamine), Polyquaternium-22 (copolymer of acrylic acid and diallyldimethylammonium chloride), Polyquaternium-24 (auaternary ammonium salt of hydroxyethyl cellulose reacted with a lauryl dimethyl ammonium substituted epoxide), Polyquaternium-27 (block copolymer of Polyquaternium-2 and Polyquaternium-17), Polyquaternium-28 (copolymer of vinylpyrrolidone and methacrylamidopropyl trimethylammonium), Polyquaternium-29 (chitosan modified with propylen oxide and quaternized with epichlorhydrin), Polyquaternium-30 (ethanaminium, N-(carboxymethyl)-N, N-dimethyl-2-[(2-methyl-1-oxo-2-propen-1-yl)oxy]-, inner salt, polymer with methyl 2-methyl-2-propenoate), Polyquaternium-31 (N,N-dimethylaminopropyl-N-acrylamidine quaternized with diethylsulfate bound to a block of polyacrylonitrile), Polyquaternium-32 (poly(acrylamide 2-methacryloxyethyltrimethyl ammonium chloride)), Polyquaternium-33 (copolymer of trimethylaminoethylacrylate salt and acrylamide), Polyquaternium-34 (copolymer of 1,3-dibromopropane and N,N-diethyl-N′,N′-dimethyl-1,3-propanediamine), Polyquaternium-35 (methosulphate of the copolymer of methacryloyloxyethyltrimethylammonium and of methacryloyloxyethyldimethylacetylammonium), Polyquaternium-36 (copolymer of N,N-dimethylaminoethylmethacrylate and buthylmethacrylate, quaternized with dimethylsulphate), Polyquaternium-37 (poly(2-methacryloxyethyltrimethylammonium chloride)), Polyquaternium-39 (terpolymer of acrylic acid, acrylamide and diallyldimethylammonium Chloride), Polyquaternium-42 (poly[oxyethylene(dimethylimino)ethylene (dimethylimino)ethylene dichloride]), Polyquaternium-43 (copolymer of acrylamide, acrylamidopropyltrimonium chloride, 2-amidopropylacrylamide sulfonate and dimethylaminopropylamine), Polyquaternium-44 (3-Methyl-1-vinylimidazolium methyl sulfate-N-vinylpyrrolidone copolymer), Polyquaternium-45 (copolymer of (N-methyl-N-ethoxyglycine)methacrylate and N,N-dimethylaminoethylmethacrylate, quaternized with dimethyl sulphate), Polyquaternium-46 (terpolymer of vinylcaprolactam, vinylpyrrolidone, and quaternized vinylimidazole), Polyquaternium-47 (terpolymer of acrylic acid, methacrylamidopropyl trimethylammonium chloride, and methyl acrylate), and/or Polyquaternium-67.

In various embodiments, the one or more cationic conditioning polymers are chosen from cationic cellulose derivatives, quaternized hydroxyethyl cellulose (e.g., polyquaternium-10), cationic starch derivatives, cationic guar gum derivatives, copolymers of acrylamide and dimethyldiallyammonium chloride (e.g., polyquaternium-7), polyquaterniums, and a mixture thereof. For example, the cationic polymer(s) may be selected from polyquaterniums, for example, polyquaterniums selected from polyquaternium-4, polyquaternium-5, polyquaternium-6, polyquaternium-7, polyquaternium-10, polyquaternium-22, polyquaternium-37, polyquaternium-39, polyquaternium-47, polyquaternium-53, polyquaternium-67 and a mixture thereof. A combination of two or more polyquaterniums can be useful. A particularly preferred and useful cationic polymer is polyquaternium-10.

The cationic polymers may be a polyquaternium. In certain embodiments, the cationic surfactants may be polyquaterniums selected from polyquaternium-1, polyquaternium-2, polyquaternium-3, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-21, polyquaternium-22, polyquaternium-23, polyquaternium-24, polyquaternium-25, polyquaternium-26, polyquaternium-27, polyquaternium-28, polyquaternium-29, polyquaternium-30, polyquaternium-40, polyquaternium-41, polyquaternium-42, polyquaternium-43, polyquaternium-44, polyquaternium-45, polyquaternium-46, polyquaternium-47, polyquaternium-48, polyquaternium-49, polyquaternium-50, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-61, polyquaternium-62, polyquaternium-63, polyquaternium-64, polyquaternium-65, polyquaternium-66, polyquaternium-67, etc. In some cases, preferred polyquaternium compounds include polyquaternium-10, polyquaternium-11, polyquaternium-67, and a mixture thereof.

In some embodiments, the one or more cationic conditioning polymers are chosen from cationic proteins and cationic protein hydrolysates (e.g., hydroxypropyltrimonium hydrolyzed wheat protein), quaternary diammonium polymers (e.g., hexadimethrine chloride), copolymers of acrylamide and dimethyldiallyammonium chloride, and mixtures thereof.

The cationic conditioning polymers may be homopolymers or formed from two or more types of monomers. The molecular weight of the polymer may be between 5,000 and 10,000,000, typically at least 10,000, and preferably in the range 100,000 to about 2,000,000. These polymers will typically have cationic nitrogen containing groups such as quaternary ammonium or protonated amino groups, or a mixture thereof.

The cationic charge density is suitably at least 0.1 meq/g, preferably above 0.8 or higher. In some instances, the cationic charge density does not exceed 3 meq/g, or does not exceed 2 meq/g. The charge density can be measured using the Kjeldahl method and can be within the above limits at the desired pH of use, which will in general be from about 3 to 9 and preferably between 4 and 8.

The cationic nitrogen-containing group will generally be present as a substituent on a fraction of the total monomer units of the cationic conditioning polymer. Thus when the polymer is not a homopolymer it can contain spacer non-cationic monomer units.

Suitable cationic conditioning polymers include, for example, copolymers of vinyl monomers having cationic amine or quaternary ammonium functionalities with water soluble spacer monomers such as (meth)acrylamide, alkyl and dialkyl (meth)acrylamides, alkyl (meth)acrylate, vinyl caprolactone and vinyl pyrrolidine. The alkyl and dialkyl substituted monomers preferably have C₁-C₇ alkyl groups, more preferably C₁-C₃ alkyl groups. Other suitable spacers include vinyl esters, vinyl alcohol, maleic anhydride, propylene glycol and ethylene glycol.

The cationic amines can be primary, secondary or tertiary amines, depending upon the particular species and the pH of the composition.

Amine substituted vinyl monomers and amines can be polymerized in the amine form and then converted to ammonium by quaternization.

Suitable cationic amino and quaternary ammonium monomers include, for example, vinyl compounds substituted with dialkyl amincalkyl acrylate, dialkylamino alkylmethacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidine, e.g., alkyl vinyl imidazolium, and quaternized pyrrolidine, e.g., alkyl vinyl imidazolium, alkyl vinyl pyridinium, alkyl vinyl pyrrolidine salts. The alkyl portions of these monomers are preferably lower alkyls such as the C₁-C₃ alkyls, more preferably C₁ and C₂ alkyls.

Suitable amine-substituted vinyl monomers include dialkylaminoalkyl acrylate, dialkylaminoalkyl methacrylate, dialkylaminoalkyl acrylamide, and dialkylaminoalkyl methacrylamide, wherein the alkyl groups are preferably C₁-C₇ hydrocarbyls, more preferably C₁-C₃, alkyls.

The cationic conditioning polymers can comprise mixtures of monomer units derived from amine- and/or quaternary ammonium-substituted monomer and/or compatible spacer monomers.

Suitable cationic conditioning polymers include, for example: copolymers of 1-vinyl-2-pyrrolidine and 1-vinyl-3-methyl-imidazolium salt (e.g., Chloride salt) (referred to as Polyquaternium-16) such as those commercially available from BASF under the LUVIQUAT tradename (e.g., LUVIQUAT FC 370); copolymers of 1-vinyl-2-pyrrolidine and dimethylaminoethyl methacrylate (referred to as Polyquaternium-11) such as those commercially from Gar Corporation (Wayne, N.J., USA) under the GAFQUAT tradename (e.g., GAFQUAT 755N); and cationic diallyl quaternary ammonium-containing polymer including, for example, dimethyldiallyammonium chloride homopolymer and copolymers of acrylamide and dimethyldiallyammonium chloride (referred to as Polyquaternium-6 and Polyquaternium-7).

Other cationic conditioning polymers that can be used include polysaccharide polymers, such as cationic cellulose derivatives and cationic starch derivatives. Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide (referred to as Polyquaternium-10). Another type of cationic cellulose includes the polymeric quaternary ammonium salts of hydroxyethyl cellulose reacted with lauryl dimethyl ammonium-substituted epoxide (referred to as Polyquaternium-24). These materials are available from Amerchol Corp. (Edison, N.J., USA) under the tradename Polymer LM-200.

Other cationic conditioning polymers that can be used include cationic guar gum derivatives, such as guar hydroxypropyltrimonium chloride.

Polyquaterniums include Polyquaternium-1 (ethanol, 2,2′,2 ″-nitrilotris-, polymer with 1,4-dichloro-2-butene and N, N, N′,N′-tetramethyl-2-butene-1,4-diamine), Polyquaternium-2, (poly[bis(2-chloroethyl) ether-alt-1,3-bis[3-(dimethylamino)propyl]urea]), Polyquaternium-4, (hydroxyethyl cellulose dimethyl diallylammonium chloride copolymer; Diallyldimethylammonium chloride-hydroxyethyl cellulose copolymer), Polyquaternium-5 (copolymer of acrylamide and quaternized dimethylammoniumethyl methacrylate), polyquaternium-6 (poly(diallyldimethylammonium chloride)), Polyquaternium-7 (copolymer of acrylamide and diallyldimethylammonium chloride), Polyquaternium-8 (copolymer of methyl and stearyl dimethylaminoethyl ester of methacrylic acid, quaternized with dimethylsulphate), Polyquaternium-9 (homopolymer of N, N-(dimethylamino)ethyl ester of methacrylic acid, quaternized with bromomethane), Polyquaternium-10 (quaternized hydroxyethyl cellulose), polyquaternium-11 (copolymer of vinylpyrrolidone and quaternized dimethylaminoethyl methacrylate), Polyquaternium-12 (ethyl methacrylate/abietyl methacrylate/diethylaminoethyl methacrylate copolymer quaternized with dimethyl sulfate), Polyquaternium-13 (ethyl methacrylate/oleyl methacrylate/diethylaminoethyl methacrylate copolymer quaternized with dimethyl sulfate), Polyquaternium-14 (trimethylaminoethylmethacrylate homopolymer), Polyquaternium-15 (acrylamide-dimethylaminoethyl methacrylate methyl chloride copolymer), Polyquaternium-16 (copolymer of vinylpyrrolidone and quaternized vinylimidazole), Polyquaternium-17 (adipic acid, dimethylaminopropylamine and dichloroethylether copolymer), Polyquaternium-18 (azelanic acid, dimethylaminopropylamine and dichloroethylether copolymer), Polyquaternium-19 (copolymer of polyvinyl alcohol and 2,3-epoxypropylamine), Polyquaternium-20 (copolymer of polyvinyl octadecyl ether and 2,3-epoxypropylamine), Polyquaternium-22 (copolymer of acrylic acid and diallyldimethylammonium chloride), Polyquaternium-24 (auaternary ammonium salt of hydroxyethyl cellulose reacted with a lauryl dimethyl ammonium substituted epoxide), Polyquaternium-27 (block copolymer of Polyquaternium-2 and Polyquaternium-17), Polyquaternium-28 (copolymer of vinylpyrrolidone and methacrylamidopropyl trimethylammonium), Polyquaternium-29 (chitosan modified with propylen oxide and quaternized with epichlorhydrin), Polyquaternium-30 (ethanaminium, N-(carboxymethyl)-N, N-dimethyl-2-[(2-methyl-1-oxo-2-propen-1-yl)oxy]-, inner salt, polymer with methyl 2-methyl-2-propenoate), Polyquaternium-31 (N,N-dimethylaminopropyl-N-acrylamidine quaternized with diethylsulfate bound to a block of polyacrylonitrile), Polyquaternium-32 (poly(acrylamide 2-methacryloxyethyltrimethyl ammonium chloride)), Polyquaternium-33 (copolymer of trimethylaminoethylacrylate salt and acrylamide), Polyquaternium-34 (copolymer of 1,3-dibromopropane and N,N-diethyl-N′,N′-dimethyl-1,3-propanediamine), Polyquaternium-35 (methosulphate of the copolymer of methacryloyloxyethyltrimethylammonium and of methacryloyloxyethyldimethylacetylammonium), Polyquaternium-36 (copolymer of N,N-dimethylaminoethylmethacrylate and buthylmethacrylate, quaternized with dimethylsulphate), Polyquaternium-37 (poly(2-methacryloxyethyltrimethylammonium chloride)), Polyquaternium-39 (terpolymer of acrylic acid, acrylamide and diallyldimethylammonium Chloride), Polyquaternium-42 (poly[oxyethylene(dimethylimino)ethylene (dimethylimino)ethylene dichloride]), Polyquaternium-43 (copolymer of acrylamide, acrylamidopropyltrimonium chloride, 2-amidopropylacrylamide sulfonate and dimethylaminopropylamine), Polyquaternium-44 (3-Methyl-1-vinylimidazolium methyl sulfate-N-vinylpyrrolidone copolymer), Polyquaternium-45 (copolymer of (N-methyl-N-ethoxyglycine)methacrylate and N, N-dimethylaminoethylmethacrylate, quaternized with dimethyl sulphate), Polyquaternium-46 (terpolymer of vinylcaprolactam, vinylpyrrolidone, and quaternized vinylimidazole), and Polyquaternium-47 (terpolymer of acrylic acid, methacrylamidopropyl trimethylammonium chloride, and methyl acrylate).

The total amount of the one or more cationic conditioning polymers in the hair treatment compositions, if present, will vary. Nonetheless, in various embodiments, the hair treatment composition includes about 0.1 to about 8 wt. % of the one or more cationic conditioning polymers, based on the total weight of the hair treatment composition. In further embodiments, the hair treatment composition includes about about 0.1 to about 6 wt. %, about 0.1 to about 5 wt. %, about 0.1 to about 4 wt. %, about 0.1 to about 3 wt. %, about 0.5 to about 8 wt. %, about 0.5 to about 6 wt. %, about 0.5 to about 5 wt. %, about 0.5 to about 4 wt. %, about 0.5 to about 3 wt. %, or about 0.5 to about 2 wt. %, based on the one or more cationic conditioning polymers, based on a total weight of the hair treatment composition.

### (d) Fatty Alcohols

The term “fatty alcohol” means an alcohol comprising at least one hydroxyl group (OH), and comprising at least 8 carbon atoms, and which is neither oxyalkylenated (in particular neither oxyethylenated nor oxypropylenated) nor glycerolated. The fatty alcohols can be represented by: R—OH, wherein R denotes a saturated (alkyl) or unsaturated (alkenyl) group, linear or branched, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

The fatty alcohols may be liquid or solid. In some instances, it is preferable that the hair treatment compositions include at least one solid fatty alcohol. The solid fatty alcohols that may be used include those that are solid at ambient temperature and at atmospheric pressure (25° C., 780 mmHg), and are insoluble in water, that is to say they have a water solubility of less than 1% by weight, preferably less than 0.5% by weight, at 25° C., 1 atm.

The solid fatty alcohols may be represented by: R—OH, wherein R denotes a linear alkyl group, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

Non-limiting examples of useful fatty alcohols include lauryl alcohol or lauryl alcohol (1-dodecanol); myristic or myristyl alcohol (1-tetradecanol); cetyl alcohol (1-hexadecanol); stearyl alcohol (1-octadecanol); arachidyl alcohol (1-eicosanol); behenyl alcohol (1-docosanol); lignoceryl alcohol (1-tetracosanol); ceryl alcohol (1-hexacosanol); montanyl alcohol (1-octacosanol); myricylic alcohol (1-triacontanol), and mixtures thereof.

Preferably, the solid fatty alcohol is chosen from cetyl alcohol, stearyl alcohol, behenyl alcohol and mixtures thereof such as cetylstearyl or cetearyl alcohol.

The liquid fatty alcohols, in particular those containing C₁₀-C₃₄, preferably have branched carbon chains and/or have one or more, preferably 1 to 3 double bonds. They are preferably branched and/or unsaturated (C═C double bond) and contain from 12 to 40 carbon atoms.

The liquid fatty alcohols may be represented by: R—OH, wherein R denotes a C12-C24 branched alkyl group or an alkenyl group (comprising at least one C₁₂-C₂₄ double bond C═C), R being optionally substituted by a or more hydroxy groups. Preferably, the liquid fatty alcohol is a branched saturated alcohol. Preferably, R does not contain a hydroxyl group. These include oleic alcohol, linoleic alcohol, linolenic alcohol, isocetyl alcohol, isostearyl alcohol, 2-octyl-1-dodecanol, 2-butyloctanol, 2-hexyl-1-decanol, 2-decyl-1-tetradecanol, 2-tetradecyl-1-cetanol and mixtures thereof. Preferably, the liquid fatty alcohol is 2-octyl-1-dodecanol.

In some instances, the hair treatment compositions include one or more fatty alcohols selected from decyl alcohol, undecyl alcohol, dodecyl, myristyl, cetyl alcohol, stearyl alcohol, cetearyl alcohol, isostearyl alcohol, isocetyl alcohol, behenyl alcohol, linalool, oleyl alcohol, myricyl alcohol and a mixture thereof. In some instances, the hair cosmetic compositions preferably include cetearyl alcohol.

The total amount of the one or more fatty alcohols in the hair treatment composition will vary. Nonetheless, in various embodiments, the hair treatment composition includes about 1 to about 15 wt. % of the one or more fatty alcohols, based on the total weight of the hair treatment composition. In further embodiments, the hair treatment composition includes about 1 to about 12 wt. %, about 1 to about 10 wt. %, about 2 to about 15 wt. %, about 2 to about 12 wt. %, about 2 to about 10 wt. %, about 3 to about 15 wt. %, about 3 to about 12 wt. %, about 3 to about 10 wt. %, about 5 to 15 wt. %, about 5 to about 12 wt. %, or about 5 to about 10 wt. % of the one or more fatty alcohols, based on the total weight of the hair treatment composition.

### (e) Water

The amount of water in the hair treatment composition will vary. Nonetheless, in various embodiments the hair treatment composition includes about 55 to about 95 wt. % water. In further embodiments, the hair treatment composition includes about 55 to about 90 wt. %, about 55 to about 80 wt. %, about 55 to about 75 wt. %, about 60 to about 95 wt. %, about 60 to about 90 wt. %, about 60 to about 85 wt. %, about 60 to about 80 wt. %, about 60 to about 75 wt. %, about 65 to about 85 wt. %, about 65 to about 80 wt. %, or about 65 to about 75 wt. %, based on a total weight of the hair treatment composition.

### (f) Nonionic Thickening Polymers

The hair treatment compositions optionally include one or more nonionic thickening polymers (also referred to as “nonionic thickening agents” “rheology modifiers,” “thickening compounds,” “thickeners,” “gelling agents,” and the like. Nonlimiting examples of nonionic thickening polymers include nonionic guar gums, sclerotium gum, biopolysaccharide gums of microbial origin, gums derived from plant exudates, celluloses, in particular hydroxypropylcelluloses or hydroxyethylcelluloses, pectins, and mixtures thereof.

Suitable nonionic thickening polymers include celluloses modified with groups comprising at least one fatty chain. Examples of such modified celluloses may include: hydroxyethylcelluloses modified with groups comprising at least one fatty chain, such as alkyl, arylalkyl or alkylaryl groups, or mixtures thereof, and in which the alkyl groups are preferably C₈-C₂₂, for instance the product NATROSOL PLUS GRADE 330 CS (016 alkyls) sold by the company Aqualon, or the product BERMOCOLL EHM 100 sold by the company Berol Nobel; and hydroxyethylcelluloses modified with alkylphenyl polyalkylene glycol ether groups, such as the product AMERCELL POLYMER HM-1500 (polyethylene glycol (15) nonylphenyl ether) sold by the company Amerchol,

Further suitable nonionic thickening polymers include hydroxypropyl guars modified with groups comprising at least one fatty chain, such as the product ESAFLOR HM 22 (C₂₂ alkyl chain) sold by the company Lamberti, and the products RE210-18 (C₁₄ alkyl chain) and RE205-1 (C₂₀ alkyl chain) sold by the company Rhone-Poulenc.

Still other suitable nonionic thickening polymers may include copolymers of vinylpyrrolidone and of fatty-chain hydrophobic monomers; copolymers of 01-06 alkyl methacrylates or acrylates and of amphiphilic monomers comprising at least one fatty chain; polyurethane polyethers comprising in their chain both hydrophilic blocks usually of polyoxyethylenated nature and hydrophobic blocks, which may be aliphatic sequences alone and/or cycloaliphatic and/or aromatic sequences; or polymers with an aminoplast ether backbone containing at least one fatty chain.

In some instances, the nonionic thickening polymers may be selected from polysaccharides and associative polymers. In some cases, the preferred nonionic thickening agents are sclerotium gum, guar gums, hydroxyalkyl celluloses optionally modified with a hydrophobic group, such as hydroxyethylcelluloses, hydroxymethylcelluloses optionally modified with a hydrophobic group, and inulins optionally modified with a hydrophobic group. In some cases, sclerotium gum is particularly useful.

Among the nonionic thickening polymers that may be mentioned are:
(1) Celluloses modified with groups comprising at least one fatty chain; examples that may be mentioned include: hydroxyethylcelluloses modified with groups comprising at least one fatty chain, such as alkyl, arylalkyl or alkylaryl groups, or mixtures thereof, and in which the alkyl groups are preferably C8-C22, for instance the product NATROSOL PLUS GRADE 330 CS (C₁₆ alkyls) sold by the company Aqualon, or the product BERMOCOLL EHM 100 sold by the company Berol Nobel; and hydroxyethylcelluloses modified with alkylphenyl polyalkylene glycol ether groups, such as the product AMERCELL POLYMER HM-1500 (polyethylene glycol (15) nonylphenyl ether) sold by the company Amerchol,
(2) Hydroxypropyl guars modified with groups comprising at least one fatty chain, such as the product ESAFLOR HM 22 (C₂₂ alkyl chain) sold by the company Lamberti, and the products RE210-18 (C₁₄ alkyl chain) and RE205-1 (C₂₀ alkyl chain) sold by the company Rhone-Poulenc,
(3) Copolymers of vinylpyrrolidone and of fatty-chain hydrophobic monomers; examples that may be mentioned include: the products ANTARON V216 or GANEX V216 (vinylpyrrolidone/hexadecene copolymer) sold by the company I.S.P. the products ANTARON V220 or GANEX V220 (vinylpyrrolidone/eicosene copolymer) sold by the company I.S.P.,
(4) Copolymers of C₁-C₆ alkyl methacrylates or acrylates and of amphiphilic monomers comprising at least one fatty chain, for instance the oxyethylenated methyl acrylate/stearyl acrylate copolymer sold by the company Goldschmidt under the name ANTIL 208,
(5) Copolymers of hydrophilic methacrylates or acrylates and of hydrophobic monomers comprising at least one fatty chain, for instance the polyethylene glycol methacrylate/lauryl methacrylate copolymer,
(6) Polyurethane polyethers comprising in their chain both hydrophilic blocks usually of polyoxyethylenated nature and hydrophobic blocks, which may be aliphatic sequences alone and/or cycloaliphatic and/or aromatic sequences.

In certain embodiments, at least one of the one or more thickening polymers in the cosmetic composition is a taurate copolymer.

Nonlimiting examples taurate copolymers include acrylamide/sodium acryloyl dimethyl taurate copolymer, hydroxyethyl acrylate/sodium acryloyl dimethyl taurate copolymer, ammonium acryloyldimethyl taurate/VP copolymer, sodium acrylate/sodium acryloyl dimethyl taurate copolymer, hydroxyethyl acrylate/sodium acryloyldimethyl taurate copolymer, and a mixture thereof

The amount of the one or more nonionic thickening polymers will vary depending on the type of thickening polymers used; and depending on the desired viscosity of the hair treatment composition. Therefore, the total amount of the one or more thickening agents is sufficient to achieve the viscosities set forth throughout the instant disclosure. Nonetheless, in various embodiments, the hair treatment compositions include about 0.01 to about 8 wt. %, based on the total weight of the cosmetic composition. In further embodiments, the total amount of the one or more thickening agents is from about 0.01 to about 6 wt. %, about 0.01 to about 5 wt. %, about 0.01 to about 4 wt. %, about 0.01 to about 3 wt. %, about 0.1 to about 6 wt. %, about 0.1 to about 5 wt. %, about 0.1 to about 4 wt. %, about 0.5 to about 6 wt. %, about 0.5 to about 5 wt. %, or about 0.5 to about 4 wt. %, based on a total weight of the hair treatment composition.

### (g) Surfactants

The hair treatment compositions optionally include one or more surfactants, other than the cationic or cationizable surfactants mentioned above, which are characterized as cationic conditioning agents. Surfactants (other than cationic or cationizable surfactants) include amphoteric (zwitterionic) surfactants, nonionic surfactants, and anionic surfactants. Preferably, the hair treatment compositions include one or more amphoteric surfactants, nonionic surfactants, or a combination thereof. In various embodiments, the hair treatment compositions include one or more amphoteric surfactants. In various embodiments, the hair treatment compositions include one or more nonionic surfactants. The hair treatment compositions may optionally include one or more anionic surfactants but in various embodiments, the hair treatment compositions are free or essentially free from anionic surfactants.

The total amount of the one or more surfactants (other than the cationic and/or cationizable surfactants), if preset, will vary. In various embodiments, the hair treatment composition includes about 0.1 to about 10 wt. % of the one or more surfactants (other than the cationic and/or cationizable surfactants), based on a total weight of the hair treatment composition. In further embodiments, the hair treatment composition includes about 0.1 to about 8 wt. %, about 0.1 to about 6 wt. %, about 0.1 to about 5 wt. %, about 0.5 to about 10 wt. %, about 0.5 to about 8 wt. %, about 0.5 to about 6 wt. %, about 0.5 to about 5 wt. %, about 1 to about 10 wt. %, about 1 to about 8 wt. %, about 1 to about 6 wt. %, about 1 to about 5 wt. %, about 2 to about 10 wt. %, about 2 to about 8 wt. %, about 2 to about 6 wt. %, or about 2 to about 5 wt. % of the one or more surfactants (other than the cationic and/or cationizable surfactants), based on a total weight of the hair treatment composition.

### (i) Amphoteric Surfactants

Nonlimiting examples of useful amphoteric surfactants include alkyl betaines, alkyl sultaines, alkyl amphoacetates, alkyl amphoproprionates, and mixtures thereof. In a preferred embodiment, the hair treatment compositions include one or more betaines and/or alkyl betaines. Non-limiting examples of useful amphoteric surfactants are provided below.

(i-a) Alkyl Betaines

Useful betaines include those of the following formulae (XIa-XId):

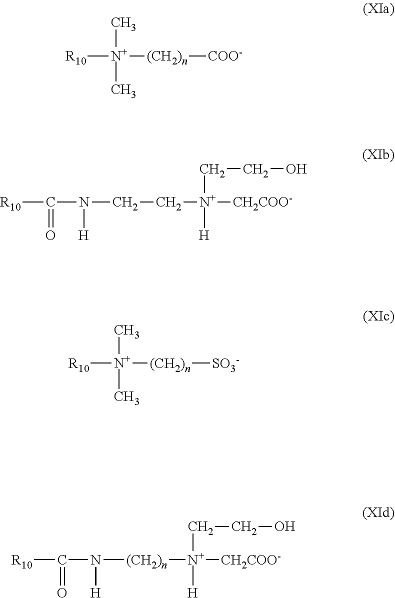

wherein R10 is an alkyl group having 8-18 carbon atoms; and n is an integer from 1 to 3.

Nonlimiting examples of useful betains include cocobetaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, and mixtures thereof. Typically, at least one betaine compound is selected from coco betaine, cocamidopropyl betaine, behenyl betaine, capryl/capramidopropyl betaine, and lauryl betaine, and mixtures thereof. Particularly preferred betaines include coco betaine and cocamidopropyl betaine.

The total amount of alkyl betaines in the hair treatment compositions composition, if present, will vary. Nonetheless, in various embodiments the hair treatment composition includes about 0.1 to about 10 wt. % of the one or more alkyl betaines, based on a total weight of the hair treatment composition. In further embodiments, the hair treatment composition includes about 0.1 to about 8 wt. %, about 0.1 to about 6 wt. %, about 0.1 to about 5 wt. %, about 0.5 to about 10 wt. %, about 0.5 to about 8 wt. %, about 0.5 to about 6 wt. %, about 0.5 to about 5 wt. %, about 1 to about 10 wt. %, about 1 to about 8 wt. %, about 1 to about 6 wt. %, about 1 to about 5 wt. %, about 2 to about 10 wt. %, about 2 to about 8 wt. %, about 2 to about 6 wt. %, or about 2 to about 5 wt. % of the one or more alkyl betaines, based on a total weight of the hair treatment composition.

(i-b) Alkyl Sultaines

Non-limiting examples of alkyl sulitaines include hydroxyl sultaines of formula (XII)

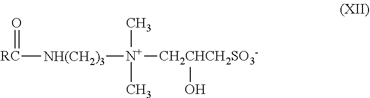

wherein R is an alkyl group having 8-18 carbon atoms. More specific examples include, but are not limited to cocamidopropyl hydroxysultaine, lauryl hydroxysultaine, and a mixture thereof.

The total amount of alkyl sulitaines in the hair treatment compositions composition, if present, will vary. Nonetheless, in various embodiments the hair treatment composition includes about 0.1 to about 10 wt. % of the one or more alkyl sulitaines, based on a total weight of the hair treatment composition. In further embodiments, the hair treatment composition includes about 0.1 to about 8 wt. %, about 0.1 to about 6 wt. %, about 0.1 to about 5 wt. %, about 0.5 to about 10 wt. %, about 0.5 to about 8 wt. %, about 0.5 to about 6 wt. %, about 0.5 to about 5 wt. %, about 1 to about 10 wt. %, about 1 to about 8 wt. %, about 1 to about 6 wt. %, about 1 to about 5 wt. %, about 2 to about 10 wt. %, about 2 to about 8 wt. %, about 2 to about 6 wt. %, or about 2 to about 5 wt. % of the one or more alkyl sulitaines, based on a total weight of the hair treatment composition.

(i-c) Alkyl Amphoacetates and Alkyl Amphodiacetates

Nonlimiting examples of useful alkyl amphoacetates and alkyl amphodiacetates include those of Formula (XIII) and (XIV):

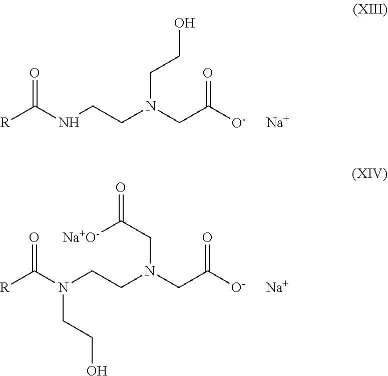

wherein R is an alkyl group having 8-18 carbon atoms. Sodium is shown as the cation in the above formulae above but the cation may be an alkali metal ion such as sodium or potassium, ammonium ions, or alkanolammonium ions such as monoethanolammonium or triethanolammonium ions. A more specific, but non-limiting example, is sodium lauroamphoacetate.

The total amount of alkyl amphoacetates and alkyl amphodiacetates in the hair treatment compositions composition, if present, will vary. Nonetheless, in various embodiments the hair treatment composition includes about 0.1 to about 10 wt. % of the one or more alkyl amphoacetates and alkyl amphodiacetates, based on a total weight of the hair treatment composition. In further embodiments, the hair treatment composition includes about 0.1 to about 8 wt. %, about 0.1 to about 6 wt. %, about 0.1 to about 5 wt. %, about 0.5 to about 10 wt. %, about 0.5 to about 8 wt. %, about 0.5 to about 6 wt. %, about 0.5 to about 5 wt. %, about 1 to about 10 wt. %, about 1 to about 8 wt. %, about 1 to about 6 wt. %, about 1 to about 5 wt. %, about 2 to about 10 wt. %, about 2 to about 8 wt. %, about 2 to about 6 wt. %, or about 2 to about 5 wt. % of the one or more alkyl amphoacetates and alkyl amphodiacetates, based on a total weight of the hair treatment composition.

(i-d) Alkyl Amphopropionates

Nonlimiting examples of useful alkyl amphopropionates include cocoampho-propionate, cornamphopropionatecaprylamphopropionate, cornampho-propionate, caproamphopropionate, oleoamphopropionate, isostearoamphopropionate, stearoamphopropionate, lauroamphopropionate, salts thereof, and a mixture thereof.

The total amount of alkyl amphopropionates in the hair treatment compositions composition, if present, will vary. Nonetheless, in various embodiments the hair treatment composition includes about 0.1 to about 10 wt. % of the one or more alkyl amphopropionates, based on a total weight of the hair treatment composition. In further embodiments, the hair treatment composition includes about 0.1 to about 8 wt. %, about 0.1 to about 6 wt. %, about 0.1 to about 5 wt. %, about 0.5 to about 10 wt. %, about 0.5 to about 8 wt. %, about 0.5 to about 6 wt. %, about 0.5 to about 5 wt. %, about 1 to about 10 wt. %, about 1 to about 8 wt. %, about 1 to about 6 wt. %, about 1 to about 5 wt. %, about 2 to about 10 wt. %, about 2 to about 8 wt. %, about 2 to about 6 wt. %, or about 2 to about 5 wt. % of the one or more alkyl amphopropionates, based on a total weight of the hair treatment composition.

### (ii) Nonionic Surfactants

The hair treatment composition may optionally include or exclude (or be essentially free from) one or more nonionic surfactants. Non-limiting examples of nonionic surfactants include the following:
(1) polyethylene oxide condensates of alkyl phenols, e.g., the condensation products of alkyl phenols having an alkyl group containing from about 6 to about 20 carbon atoms in either a straight chain or branched chain configuration, with ethylene oxide, the said ethylene oxide being present in amounts equal to from about 10 to about 60 moles of ethylene oxide per mole of alkyl phenol;
(2) those derived from the condensation of ethylene oxide with the product resulting from the reaction of propylene oxide and ethylene diamine products;
(3) condensation products of aliphatic alcohols having from about 8 to about 18 carbon atoms, in either straight chain or branched chain configurations, with ethylene oxide, e.g., a coconut alcohol ethylene oxide condensate having from about 10 to about 30 moles of ethylene oxide per mole of coconut alcohol, the coconut alcohol fraction having from about 10 to about 14 carbon atoms;
(4) long chain tertiary amine oxides of the formula [R¹R²R³N→O] where R¹ contains an alkyl, alkenyl or monohydroxy alkyl radical of from about 8 to about 18 carbon atoms, from 0 to about 10 ethylene oxide moieties, and from 0 to about 1 glyceryl moiety, and R² and R³ contain from about 1 to about 3 carbon atoms and from 0 to about 1 hydroxy group, e.g., methyl, ethyl, propyl, hydroxyethyl, or hydroxypropyl radicals;
(5) long chain tertiary phosphine oxides of the formula [RR′R″P→O] where R contains an alkyl, alkenyl or monohydroxyalkyl radical ranging from about 8 to about 18 carbon atoms in chain length, from 0 to about 10 ethylene oxide moieties and from 0 to 1 glyceryl moieties and R′ and R″ are each alkyl or monohydroxyalkyl groups containing from about 1 to about 3 carbon atoms;
(6) long chain dialkyl sulfoxides containing one short chain alkyl or hydroxy alkyl radical of from 1 to about 3 carbon atoms (usually methyl) and one long hydrophobic chain which include alkyl, alkenyl, hydroxy alkyl, or keto alkyl radicals containing from about 8 to about 20 carbon atoms, from 0 to about 10 ethylene oxide moieties and from 0 to 1 glyceryl moieties;
(7) alkyl polysaccharide (APS) surfactants (e.g. alkyl polyglycosides), including APS surfactants having a hydrophobic group with about 6 to about 30 carbon atoms and a polysaccharide (e.g., polyglycoside) as the hydrophilic group; optionally, there can be a polyalkylene-oxide group joining the hydrophobic and hydrophilic moieties; and the alkyl group (i.e., the hydrophobic moiety) can be saturated or unsaturated, branched or unbranched, and unsubstituted or substituted (e.g., with hydroxy or cyclic rings); a preferred material is alkyl polyglucoside, which is commercially available from Henkel, ICI Americas, and Seppic; and
(8) polyoxyethylene alkyl ethers such as those of the formula RO(CH₂CH₂O)ₙH and polyethylene glycol (PEG) glyceryl fatty esters, such as those of the formula R(O)OCH₂CH(OH)CH₂(OCH₂CH₂)ₙOH, wherein n is from 1 to about 200, preferably from about 20 to about 100, and R is an alkyl having from about 8 to about 22 carbon atoms.
polyethylene glycol derivatives of glycerides as described in the above (8) useful herein include derivatives of mono-, di- and tri-glycerides and mixtures thereof. One class of polyethylene glycol derivatives of glycerides suitable herein is those which conform to the general formula (I):

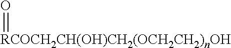

wherein n, the degree of ethoxylation, is from about 4 to about 200, preferably from about 5 to about 150, more preferably from about 20 to about 120, and wherein R comprises an aliphatic radical having from about 5 to about 25 carbon atoms, preferably from about 7 to about 20 carbon atoms. Suitable polyethylene glycol derivatives of glycerides can be polyethylene glycol derivatives of hydrogenated castor oil. Such polyethylene glycol derivatives of hydrogenated castor oil include, for example, PEG-20 hydrogenated castor oil, PEG-30 hydrogenated castor oil, PEG-40 hydrogenated castor oil, PEG-45 hydrogenated castor oil, PEG-50 hydrogenated castor oil, PEG-54 hydrogenated castor oil, PEG-55 hydrogenated castor oil, PEG-60 hydrogenated castor oil, PEG-80 hydrogenated castor oil, and PEG-100 hydrogenated castor oil.

Other suitable polyethylene glycol derivatives of glycerides can be polyethylene glycol derivatives of stearic acid. Such polyethylene glycol derivatives of stearic acid include, for example, PEG-30 stearate, PEG-40 stearate, PEG-50 stearate, PEG-75 stearate, PEG-90 stearate, PEG-100 stearate, PEG-120 stearate, and PEG-150 stearate.

Ethylene glycol ethers of fatty alcohols, as described in the above (3) or (8), useful herein include any ethylene glycol ethers of fatty alcohols which are suitable for use in a hair conditioning composition. No limiting examples of the ethylene glycol ethers of fatty alcohols include; the ceteth series of compounds such as ceteth-1 through ceteth-45, preferably ceteth-7 through ceteth-20; the isoceteth series of compounds such as isoceteth-20; the steareth series of compounds such as steareth-1 through 100; ceteareth 1 through ceteareth-50; the laureth series of compounds, preferably laureth-7 through Laureth-12; the pareth series of compounds, preferably pareth-9 through pareth-15; propylene glycol ethers of the above ceteth, steareth, ceteareth, and laureth series of compounds, such propylene glycol ethers of ceteth series of compounds including, for example, PPG-5-Ceteth-20; polyoxyethylene ethers or polyoxyethylene-polyoxypropylene ethers of branched alcohols, such branched alcohols including, for example, octyldodecyl alochol, decyltetradecyl alcohol, dodecylpentadecyl alcohol, hexyldecyl alcohol, and isostearyl alcohol, and such polyoxyethylene-polyoxypropylene ethers of branched alcohols including, for example, POE(20)POP(6) decyltetradecyl ether; and mixtures thereof.

Other nonionic surfactants useful herein include, for example, polysorbates such as polysorbate-20 (POE(20) sorbitan monolaurate) having HLB value of 16.7, polysorbate-21 (POE(4) sorbitan monolaurate) having HLB value of 13.3, polysorbate-40 (POE(20) sorbitan monopalmitate) having HLB value of 15.6, polysorbate-60 (POE(20) sorbitan monostearate) having HLB value of 14.9, polysorbate-61 (POE(4) sorbitan monostearate) having HLB value of 9.6, polysorbate-80 (POE(20)sorbitan monooleate) having HLB value of 15.0, and polysorbate-81 (POE(4) sorbitan monooleate) having HLB value of 10.0.

In one embodiment, one or more nonionic surfactants are selected from the group consisting of PEG-40 hydrogenated castor oil, steareth-2, steareth-20, polysorbate 60, polyclyceryl-3 stearate, glyceryl stearate citrate, and a mixture thereof. In some instances, the one or more nonionic surfactants includes PEG-40 hydrogenated castor oil. In one embodiment, the one or more nonionic surfactants include alkoxylated fatty alcohols or polyethylene glycol ethers of mixtures of C₈-C₃₀ fatty alcohols with an average of number of moles of ethylene oxide such as C₁₁-15 Pareth-7, laureth-9, laureth-12, deceth-7, deceth-10, trideceth-6, trideceth-10, trideceth-12, or a mixture thereof.

The total amount of the one or more nonionic surfactants in the hair treatment composition, if present, will vary. In various embodiments, the hair treatment composition includes about 0.01 to about 10 wt. % of the one or more nonionic surfactants, based on a total weight of the hair styling composition. In further embodiments, the hair treatment composition includes about 0.01 to about 8 wt. %, about 0.01 to about 6 wt. %, about 0.01 to about 5 wt. %, about 0.01 to about 3 wt. %, about 0.05 to about 10 wt. %, about 0.05 to about 8 wt. %, about 0.05 to about 6 wt. %, about 0.05 to about 5 wt. %, about 0.05 to about 3 wt. %, about 0.1 to about 10 wt. %, about 0.1 to about 8 wt. %, about 0.1 to about 6 wt. %, about 0.1 to about 5 wt. %, about 0.1 to about 3 wt. %, based on a total weight of the hair treatment composition.

### (h) Amino-Functionalized Silicone Oils

The term “amino-functionalized silicone” or “amino silicones” means a silicone containing at least one primary amino, secondary amino, tertiary amino and/or quaternary ammonium group. The structure of the amino-functionalized silicone may be linear or branched, cyclic or non-cyclic. The amino functional group may be at any position in the silicone molecule, preferably at the end of the backbone (for example, in the case of amodimethicones) and/or in the side chain.

In some instances, an amino-functionalized silicones is selected from compounds having the following formula:

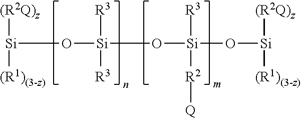

wherein each R¹ is independently selected from a C₁₋₃₀ alkyl group, a C₁₋₃₀ alkoxy group, a C₅₋₃₀ aryl group, a C₆₋₃₀ aralkyl group, a C₆₋₃₀ aralkyloxy group, a C₁₋₃₀ alkaryl group, a C₁₋₃₀ alkoxyaryl group, and a hydroxy group (preferably, each R¹ is independently selected from a C₁₋₃₀ alkyl group, a C₁₋₃₀ alkoxy group and a hydroxy group);
each R² is independently a divalent alkylene radical having one to ten carbon atoms (preferably, R² is a divalent alkylene radical having three to six carbon atoms);
each R³ is independently selected from a C₁₋₃₀ alkyl group, a C₅₋₃₀ aryl group, a C₆₋₃₀ aralkyl group and a C₁₋₃₀ alkaryl group (preferably, each R³ is independently selected from of a C₁₋₃₀ alkyl group);
Q is a monovalent radical selected from —NR⁴₂ and —NR⁴(CH₂)ₓNR⁴₂;
each R⁴ is independently selected from a hydrogen and a C₁₋₄ alkyl group;
x is 2 to 6;
z is 0 or 1;
n is 25 to 3,000 (preferably, 25 to 2,000; more preferably, 25 to 1,000; most preferably 25 to 500); and
m is 0 to 3,000 (preferably, 0 to 2,000; more preferably, 0 to 1,000; most preferably, 0 to 100);
with the proviso that at least 50 mol % of the total number of R¹ and R³ groups are methyl and with the proviso that when m is 0, z is 1.

Preferred R¹ groups include methyl, methoxy, ethyl, ethoxy, propyl, propoxy, isopropyl, isopropoxy, butyl, butoxy, isobutyl, isobutoxy, phenyl, xenyl, benzyl, phenylethyl, tolyl and hydoxy. Preferred R² divalent alkylene radicals include trimethylene, tetramethylene,

pentamethylene, —CH₂CH(CH₃)CH₂— and —CH₂CH₂CH(CH₃)CH₂—. Preferred R³ groups include methyl, ethyl, propyl, isopropyl, butyl, isobutyl, phenyl, xenyl, benzyl, phenylethyl and tolyl. Preferred R⁴ groups include methyl, ethyl, propyl, isopropyl, butyl and isobutyl. When z is 0, the amino-functionalized silicine has only pendant amine functional substituents in the polymer chain. When z is 1, the amino-functional silicone may have only terminal amine functional substituents (e.g., m=0) or may have both terminal and pendant amine functional substituents in the polymer chain (e.g., m>0). Preferably, n+m is 50 to 1,000. More preferably, n+m is 50 to 750. Still more preferably, n+m is 50 to 500. Most preferably, n+m is 50 to 250.

In some instances, the amino-functionalized silicones are alkoxylated and/or hydroxylated amino silicones. Suitable alkoxylated and/or hydroxylated amino silicones may be selected from compounds of the following formula:

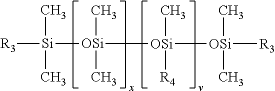

wherein R₃ is hydroxyl or OR₅, R₅ is a C₁ to C₄ alkyl group, R₄ is a group with structure according to the following formula:

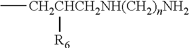

R₆ is a C₁ to C₄ alkyl, n is a 1 to 4, x is the same as “n” described above, and y is the same as “m” described above.

The silicone may be a polysiloxane corresponding to the following formula:

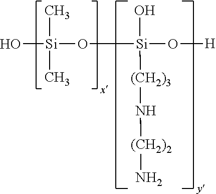

in which x′ and y′ are integers such that the weight-average molecular weight (Mw) is comprised between about 5000 and 500 000;
b) amino silicones corresponding to following formula:

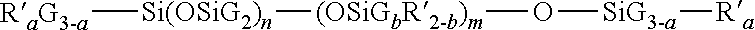

in which:
G, which may be identical or different, designate a hydrogen atom, or a phenyl, OH or C₁-C₈ alkyl group, for example methyl, or C₁-C₈ alkoxy, for example methoxy,
a, which may be identical or different, denote the number 0 or an integer from 1 to 3, in particular 0;
b denotes 0 or 1, and in particular 1;
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
R′, which may be identical or different, denote a monovalent radical having formula —CqH₂qL in which q is a number ranging from 2 to 8 and L is an optionally quaternized amino group chosen from the following groups:
—NR″-Q-N(R″)₂
—N(R″)₂
—N+(R″)₃ A-
—N+H(R″)₂ A-
—N+H₂(R″) A-
—N(R″)-Q-N+R″H₂ A-
—NR″-Q-N+(R″)₂H A-
—NR″-Q-N+(R″)₃ A-,
   in which R″, which may be identical or different, denote hydrogen, phenyl, benzyl, or a saturated monovalent hydrocarbon-based radical, for example a C₁-C₂₀ alkyl radical; Q denotes a linear or branched CrH₂ᵣ group, r being an integer ranging from 2 to 6, preferably from 2 to 4; and A- represents a cosmetically acceptable ion, in particular a halide such as fluoride, chloride, bromide or iodide.

Another group of amino silicones corresponding to this definition is represented by silicones having the following formula:

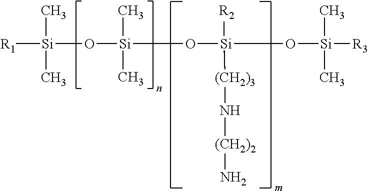

in which:
m and n are numbers such that the sum (n+m) can range from 1 to 1000, in particular from 50 to 250 and more particularly from 100 to 200, it being possible for n to denote a number from 0 to 999 and in particular from 49 to 249, and more particularly from 125 to 175, and for m to denote a number from 1 to 1000 and in particular from 1 to 10, and more particularly from 1 to 5;
R₁, R₂, R₃, which may be identical or different, represent a hydroxy or C₁-C₄ alkoxy radical, where at least one of the radicals R₁ to R₃ denotes an alkoxy radical.

The alkoxy radical is preferably a methoxy radical. The hydroxy/alkoxy mole ratio ranges preferably from 0.2:1 to 0.4:1 and preferably from 0.25:1 to 0.35:1 and more particularly equals 0.3:1. The weight-average molecular weight (Mw) of the silicone ranges preferably from 2,000 to 1,000,000, more particularly from 3,500 to 200,000.

Another group of amino silicones corresponding to this definition is represented by the following formula:

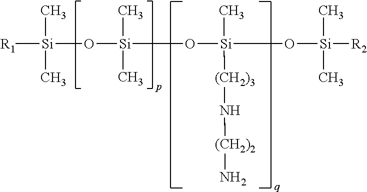

in which:
p and q are numbers such that the sum (p+q) ranges from 1 to 1000, particularly from 50 to 350, and more particularly from 150 to 250; it being possible for p to denote a number from 0 to 999 and in particular from 49 to 349, and more particularly from 159 to 239 and for q to denote a number from 1 to 1000, in particular from 1 to 10, and more particularly from 1 to 5;
R₁, R₂, which may be the same or different, represent a hydroxy or C₁-C₄ alkoxy radical, where at least one of the radicals R₁ or R₂ denotes an alkoxy radical.

The alkoxy radical is preferably a methoxy radical. The hydroxy/alkoxy mole ratio ranges generally from 1:0.8 to 1:1.1 and preferably from 1:0.9 to 1:1 and more particularly equals 1:0.95.

Another group of amino silicones is represented by the following formula:

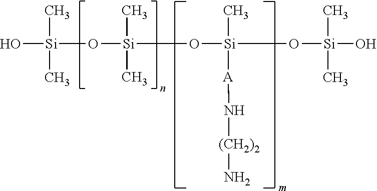

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
A denotes a linear or branched alkylene radical containing from 4 to 8 carbon atoms and preferably 4 carbon atoms. This radical is preferably linear.

The weight-average molecular weight (Mw) of these amino silicones ranges preferably from 2000 to 1 000 000 and even more particularly from 3500 to 200 000.

Another group of amino silicones is represented by the following formula:

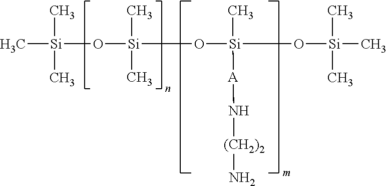

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
A denotes a linear or branched alkylene radical containing from 4 to 8 carbon atoms and preferably 4 carbon atoms. This radical is preferably branched.

The weight-average molecular weight (Mw) of these amino silicones ranges preferably from 500 to 1 000 000 and even more particularly from 1000 to 200 000. Another group of amino silicones is represented by the following formula:

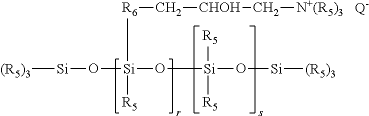

in which:
R₅ represents a monovalent hydrocarbon-based radical containing from 1 to 18 carbon atoms, and in particular a C₁-C₁₈ alkyl or C₂-C₁₈ alkenyl radical, for example methyl;
R₆ represents a divalent hydrocarbon-based radical, in particular a C₁-C₁₈ alkylene radical or a divalent C₁-C₁₈, for example C₁-C₈, alkylenoxy radical linked to the Si via an SiC bond;
Q- is an anion such as a halide ion, in particular chloride, or an organic acid salt (for example acetate);
r represents a mean statistical value from 2 to 20 and in particular from 2 to 8;
s represents a mean statistical value from 20 to 200 and in particular from 20 to 50.

Such amino silicones are described more particularly in patent U.S. Pat. No. 4,185,087.

A group of quaternary ammonium silicones is represented by the following formula:

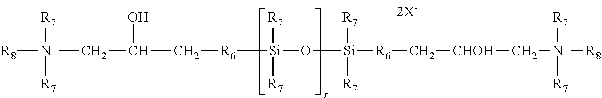

in which:
R₇, which may be identical or different, represent a monovalent hydrocarbon-based radical containing from 1 to 18 carbon atoms, and in particular a C₁-C₁₈ alkyl radical, a C₂-C₁₈ alkenyl radical or a ring containing 5 or 6 carbon atoms, for example methyl;
R₆ represents a divalent hydrocarbon-based radical, in particular a C₁-C₁₈ alkylene radical or a divalent C₁-C₁₈, for example C₁-C₈, alkylenoxy radical linked to the Si via an SiC bond;
R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl radical or a —R₆—NHCOR₇ radical;
X— is an anion such as a halide ion, in particular chloride, or an organic acid salt (for example acetate);
r represents a mean statistical value from 2 to 200 and in particular from 5 to 100. These silicones are described, for example, in patent application EP-A 0530974.

A group of quaternary ammonium silicones is represented by the following formula:

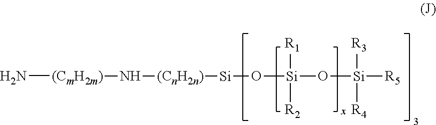

in which:
R₁, R₂, R₃ and R₄, which may be identical or different, denote a C₁-C₄ alkyl radical or a phenyl group;
R₅ denotes a C₁-C₄ alkyl radical or a hydroxyl group;
n is an integer ranging from 1 to 5;
m is an integer ranging from 1 to 5;
and in which x is chosen such that the amine number is between 0.01 and 1 meq/g;
multiblockpolyoxyalkylenated amino silicones, of type (AB)n, A being a polysiloxane block and B being a polyoxyalkylenated block containing at least one amine group.

Said silicones are preferably constituted of repeating units having the following general formulae:

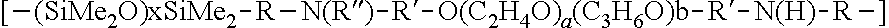

or alternatively

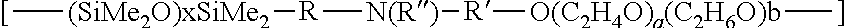

in which:
a is an integer greater than or equal to 1, preferably ranging from 5 to 200, more particularly ranging from 10 to 100;
b is an integer comprised between 0 and 200, preferably ranging from 4 to 100, more particularly between from 5 and 30;
x is an integer ranging from 1 to 10 000, more particularly from 10 to 5000;
R″ is a hydrogen atom or a methyl;
R, which may be identical or different, represent a divalent linear or branched C₂-C₁₂ hydrocarbon-based radical, optionally including one or more heteroatoms such as oxygen; preferably, R denotes an ethylene radical, a linear or branched propylene radical, a linear or branched butylene radical, or a —CH₂CH₂CH₂OCH(OH)CH₂— radical; preferentially R denotes a —CH₂CH₂CH₂OCH(OH)CH₂— radical;
R′, which may be identical or different, represent a divalent linear or branched C₂-C₁₂ hydrocarbon-based radical, optionally including one or more heteroatoms such as oxygen; preferably, R′ denotes an ethylene radical, a linear or branched propylene radical, a linear or branched butylene radical, or a —CH₂CH₂CH₂OCH(OH)CH₂— radical; preferentially R′ denotes —CH(CH₃)—CH₂—.

The siloxane blocks preferably represent between 50 and 95 mol % of the total weight of the silicone, more particularly from 70 to 85 mol %.

The amine content is preferably between 0.02 and 0.5 meq/g of copolymer in a 30% solution in dipropylene glycol, more particularly between 0.05 and 0.2. The weight-average molecular weight (Mw) of the silicone oil is preferably comprised between 5000 and 1,000,000, more particularly between 10,000 and 200,000.

Non-limiting examples of amino-functionalized silicones include bis-hydroxy/methoxy amodimethicones, bis-cetearyl amodimethicone, amodimethicone, bis(C13-15 alkoxy) PG amodimethicones, aminopropyl phenyl trimethicones, aminopropyl dimethicones, bis-amino PEG/PPG-41/3 aminoethyl PG-propyl dimethicones, caprylyl methicones, and a mixture thereof. In some instances, a particularly useful amino-functionalized silicone is bis-hydroxy/methoxy amodimethicone, wherein X is isobutyl and one of the R is OH and the other is OCH₃ in the above structure, also known as “Bis-Hydroxy/Methoxy Amodimethicone” and “3-[(2-aminoethyl)amino]-2-methylpropyl Me, di-Me, [(hydroxydimethylsilyl)oxy]- and [(methoxydimethylsilyl)oxy]-terminated.” Bis-hydroxy/methoxy amodimethicone is commercially available under the tradename DOWSIL AP-8087 FLUID from The Dow Chemical Company. A particularly preferred amino-functionalized silicone is amodimethicone” A non-limiting example of amodimethicone products containing amino silicones having structure (D) re sold by Wacker under the name BELSIL ADM 652, BELSIL ADM 4000 E, or BELSIL ADM LOG 1. A product containing amino silicones having structure (E) is sold by Wacker under the name FLUID WR 1300. Additionally or alternative, the weight-average molecular weight (Mw) of the silicone ranges preferably from 2,000 to 200,000, even more particularly 5,000 to 100,000 and more particularly from 10,000 to 50,000.

The total amount of amodimethicone in the hair treatment compositions, if present, will vary. Nonetheless, in various embodiments, the hair treatment composition includes about 0.1 to about 10 wt. % of the one or more amino-functionalized silicone, based on the total weight of the hair treatment composition. In further embodiments, the hair treatment composition includes about 0.1 to about 8 wt. %, about 0.1 to about 6 wt. %, about 0.1 to about 5 wt. %, about 0.5 to about 10 wt. %, about 0.5 to about 8 wt. %, about 0.5 to about 6 wt. %, about 0.5 to about 5 wt. %, or about 0.5 to about 4 wt. % of the one or more amino-functionalized silicones, based on a total weight of the hair treatment composition.

(h) Water-Soluble Organic Solvent Other than Glycols of (b)

The term “water soluble organic solvent” is interchangeable with the terms “water soluble solvent” and “water-miscible solvent” and means a compound that is liquid at 25° C. and at atmospheric pressure (760 mmHg), and it has a solubility of at least 50% in water under these conditions. In some cases, the water-soluble solvents has a solubility of at least 60%, 70%, 80%, or 90%. Non-limiting examples of water-soluble solvents include, for example, organic solvents selected from glycerin, alcohols (for example C₁₋₈, or C₁₋₄ alcohols), polyols (polyhydric alcohols), glycols other than the glycols of (b), and a mixture thereof.

Non-limiting examples of water soluble organic solvents include monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, ethers of propylene glycol, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water soluble organic solvents include alkanediols (polyhydric alcohols) such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

The amount of the one or more water soluble organic solvents in the hair treatment composition, if present, will vary. Nonetheless, in various embodiments, the hair treatment composition includes about 0.1 to about 10 wt. % of the one or more water soluble organic solvents, based on a total weight of the hair styling composition. In further embodiments, the hair styling composition includes about 0.1 to about 8 wt. %, about 0.1 to about 6 wt. %, about 0.1 to about 5 wt. %, about 0.5 to about 10 wt. %, about 0.5 to about 8 wt. %, about 0.5 to about 6 wt. %, about 0.5 to about 5 wt. %, about 1 to about 10 wt. %, about 1 to about 8 wt. %, about 1 to about 6 wt. %, or about 1 to about 5 wt. % of the one or more water soluble organic solvents, based on a total weight of the hair styling composition.

### (j) Miscellaneous Ingredients

The hair treatment compositions optionally include or excludes (or is essentially free from) one or more miscellaneous ingredients. In a preferred embodiment, the hair treatment composition includes one or more miscellaneous ingredients. Miscellaneous ingredients are ingredients that are compatible with the hair treatment compositions and do not disrupt or materially affect the basic and novel properties of the compositions. Nonlimiting examples of miscellaneous ingredients include preservatives, fragrances, pH adjusters, salts, chelating agents, amino acids (e.g., taurine), buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, particular materials, etc.) composition colorants, etc. In various embodiments, the miscellaneous ingredients are chosen from preservatives, fragrances, pH adjusters, salts, chelating agents, buffers, composition colorants, vegetal or botanic extracts, and mixtures thereof.

In the context of the instant disclosure, a “composition colorant” is a compound that colors the composition but does not have an appreciable coloring effect on hair. In other words, the composition colorant is included to provide a coloring to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair. Further, for purposes of the instant disclosure, pearlescent agents are considered composition colorants. Pearlescent agents are cosmetic ingredients primarily used to give a shimmering effect or pearlescent effect to the composition. They can be used to enhance the visual appeal of the hair treatment composition.

In addition to the above, the components described as optional components throughout the disclosure are can also be considered miscellaneous ingredients if not expressly set forth as an independent component of the hair treatment composition. For example, if a particular component is not expressly set forth in a claim that expressly allows for miscellaneous ingredients, such optional components should be considered included in the miscellaneous ingredients unless otherwise specified. Thus, the term “miscellaneous ingredient” is understood as a catch-all phrase representing additional components that may optionally be present in amounts designated for the miscellaneous ingredients.

The amount of the one or more miscellaneous ingredients in the hair treatment composition, if present, will vary. Nonetheless, in various embodiments, the hair treatment composition includes about 0.01 to about 10 wt. % of the one or more miscellaneous ingredients, based on a total weight of the hair treatment composition. In further embodiments, the hair treatment composition includes about 0.01 to about 8 wt. %, about 0.01 to about 6 wt. %, about 0.01 to about 5 wt. %, about 0.01 to about 3 wt. %, about 0.05 to about 10 wt. %, about 0.05 to about 8 wt. %, about 0.05 to about 6 wt. %, about 0.05 to about 5 wt. %, about 0.1 to about 10 wt. %, about 0.1 to about 8 wt. %, about 0.1 to about 6 wt. %, about 0.1 to about 5 wt. % of one or more miscellaneous ingredients, based on a total weight of the hair treatment composition.

### EMBODIMENTS

In various embodiments, the hair treatment composition is an oil-in-water composition that comprises or consists of:
(a) about 1 to about 10 wt. %, preferably about 1 to about 6, more preferably about 1.1 to about 4 wt. % of one or more polyether-modified polysiloxanes;
(b) about 2 to about 20 wt. %, preferably about 3 to about 15 wt. %, more preferably about 5 to about 12 wt. % of one or more glycols selected from ethylene glycol, propylene glycol, butylene glycol, pentylene glycol, dipropylene glycol, or a combination thereof, wherein the composition preferably includes about 2 to about 20 wt. %, preferably about 3 to about 15 wt. %, more preferably about 5 to about 12 wt. % of propylene glcyol;
(c) about 1 to about 12 wt. %, preferably about 2 to about 10 wt. %, more preferably about 2 to about 6 wt. % of one or more cationic conditioning agents, preferably wherein the hair treatment compositions includes about 1 to about 12 wt. %, preferably about 2 to about 10 wt. %, more preferably about 2 to about 6 wt. % of one or more cationic surfactants;
(d) about 1 to about 15 wt. %, preferably about 2 to about 12 wt. %, more preferably about 5 to about 10 wt. % of one or more fatty alcohols
(e) about 55 to about 85 wt. %, preferably about 60 to about 85 wt. %, more preferably about 60 to about 80 wt. % of water;
   wherein all weight percentages are based on a total weight of the composition.

In addition to the above, in further embodiments, the hair treatment optionally includes one or more of: (f) one or more nonionic thickening polymers; (g) one or more alkyl betaines; (h) one or more amino-functionalized silicone oils; (i) one or more water-soluble organic solvents; and/or (j) up to about 10 wt. % of one or more miscellaneous ingredients.

In various embodiments, the hair treatment composition is an oil-in-water composition that comprises or consists of:
(a) about 1 to about 10 wt. %, preferably about 1 to about 6, more preferably about 1.1 to about 4 wt. % of one or more polyether-modified polysiloxanes selected from PEGylated dimethicones, bis-PEGylated dimethicones, PEG/PPG dimethicones, or a combination thereof, wherein:
   the PEGylated dimethicones are selected from PEG-10 dimethicone, PEG-9 dimethicone, PEG-8 dimethicone, PEG-3 dimethicone, PEG-7 dimethicone, PEG-12 dimethicone, PEG-11 methyl ether dimethicone, PEG-32-dimethicone, PEG-14 dimethicone, PEG-17 dimethicone, or a combination thereof;
   the bis-PEGylated dimethicones are selected from bis-PEG-[10-20] dimethicone, bis-PEG-10 dimethicone, bis-PEG-12 dimethicone, bis-PEG-17 dimethicone, bis-PEG-20 dimethicone, or a combination thereof; and
   the PEG/PPG dimethicones are selected from PEG/PPG-3/10 dimethicone, PEG/PPG-4/12 dimethicone, PEG/PPG-6/11 dimethicone, PEG/PPG-8/14 dimethicone, PEG/PPG-14/4 dimethicone, PEG/PPG-15/15 dimethicone, PEG/PPG-16/2 dimethicone, PEG/PPG-17/18 dimethicone, PEG/PPG-18/18 dimethicone, PEG/PPG-19/19 dimethicone, PEG/PPG-20/6 dimethicone, PEG/PPG-20/15 dimethicone, PEG/PPG-20/20 Dimethicone, PEG/PPG-20/23 dimethicone, PEG/PPG-20/29 dimethicone, PEG/PPG-22/23 dimethicone, PEG/PPG-22/24 dimethicone, PEG/PPG-23/6 dimethicone, PEG/PPG-25/25 dimethicone, PEG/PPG-27/27, bis-PEG/PPG 18/6 dimethicone, bis-PEG/PPG-20/20 dimethicone, PEG/PPG-18/18 dimethicone, cetyl PEG/PPG-10/1 dimethicone, lauryl PEG/PPG-18/18 methicone, PEG/PPG 20/22 butyl ether dimethicone, PEG/PPG 23/6 dimethicone, PEG/PPG 20/15 dimethicone, PEG/PPG 20/23 dimethicone, or a combination thereof;
(b) about 2 to about 20 wt. %, preferably about 3 to about 15 wt. %, more preferably about 5 to about 12 wt. % of propylene glycol;
(c) about 1 to about 12 wt. %, preferably about 2 to about 10 wt. %, more preferably about 2 to about 6 wt. % of one or more cationic conditioning agents selected from cationic surfactants, cationic polymers, or a combination thereof, preferably wherein at least one of the one or more cationic conditioning agents is a cationic surfactant selected from quaternary ammonium compounds, fatty dialkylamines, fatty amidoamines, salts thereof, or a combination thereof;
(d) about 1 to about 15 wt. %, preferably about 2 to about 12 wt. %, more preferably about 5 to about 10 wt. % of one or more fatty alcohols selected from decyl alcohol, undecyl alcohol, dodecyl alcohol, myristyl alcohol, cetyl alcohol, stearyl alcohol, isostearyl alcohol, isocetyl alcohol, behenyl alcohol, linalool, oleyl alcohol, myricyl alcohol, or a mixture thereof;
(e) about 55 to about 85 wt. %, preferably about 60 to about 85 wt. %, more preferably about 60 to about 80 wt. % of water;
(f) optionally, about 0.01 to about 6 wt. %, preferably about 0.1 to about 5 wt. %, more preferably about 0.5 to about 4 wt. % of one or more nonionic thickening polymers, preferably wherein at least one of the one or more nonionic thickening polymers is selected from polysaccharides and associative polymers, in particular, sclerotium gum, guar gums, hydroxyalkyl celluloses optionally modified with a hydrophobic group, such as hydroxyethylcelluloses, hydroxymethylcelluloses optionally modified with a hydrophobic group, and inulins optionally modified with a hydrophobic group;
(g) optionally, about 0.1 to about 10 wt. %, preferably about 0.5 to about 8, more preferably about 1 to about 6 wt. % of one or more amphoteric surfactants selected from alkyl betaines, alkyl sultaines, alkyl amphoacetates, alkyl amphoproprionates, or a combination thereof, wherein preferably at least one of the one or more amphoteric surfactants is an alkyl betaine;
(h) optionally, about 0.1 to about 10 wt. %, preferably about 0.1 to about 8 wt. %, more preferably about 0.5 to about 6 wt. % of one or more amino-functionalized silicone oils, preferably amodimethicone;
(i) optionally, about 0.01 to about 10 wt. %, preferably about 0.1 to about 8 wt. %, more preferably about 0.1 to about 5 wt. % of one or more water-soluble organic solvents, preferably wherein the one or more water-soluble organic solvents are selected from mono-alcohols (for example C₁₋₆ or C₁₋₄ mono-alcohols), polyols (polyhydric alcohols), glycols other than the glycols of (b), and a mixture thereof, more preferably selected from C₁₋₆ or C₁₋₄ mono-alcohols;
(j) optionally, about 0.01 to about 10 wt. %, preferably about 0.1 to about 8 wt. %, more preferably about 1 to about 5 wt. % of one or more miscellaneous ingredients, preferably one or more miscellaneous ingredients selected from preservatives, fragrances, pH adjusters, salts, chelating agents, buffers, antioxidants, flavonoids, vitamins, amino acids (e.g., taurine), botanical extracts, UV filtering agents, proteins, protein hydrolysates, and/or isolates, fillers (e.g., organic and/or inorganic fillers such as talc, calcium carbonate, silica, particular materials, etc.) composition colorants, or a mixture thereof;
   wherein all weight percentages are based on a total weight of the composition.

In further embodiments, the hair treatment composition is an oil-in-water composition that comprises or consists of:
(a) about 1 to about 10 wt. %, preferably about 1 to about 6, more preferably about 1.1 to about 4 wt. % of one or more polyether-modified polysiloxanes selected from PEGylated dimethicones, bis-PEGylated dimethicones, PEG/PPG dimethicones, or a combination thereof, wherein:
   the PEGylated dimethicones are selected from PEG-10 dimethicone, PEG-9 dimethicone, PEG-8 dimethicone, PEG-3 dimethicone, PEG-7 dimethicone, PEG-12 dimethicone, PEG-11 methyl ether dimethicone, PEG-32-dimethicone, PEG-14 dimethicone, PEG-17 dimethicone, or a combination thereof;
   the bis-PEGylated dimethicones are selected from bis-PEG-[10-20] dimethicone, bis-PEG-10 dimethicone, bis-PEG-12 dimethicone, bis-PEG-17 dimethicone, bis-PEG-20 dimethicone, or a combination thereof; and
   the PEG/PPG dimethicones are selected from PEG/PPG-3/10 dimethicone, PEG/PPG-4/12 dimethicone, PEG/PPG-6/11 dimethicone, PEG/PPG-8/14 dimethicone, PEG/PPG-14/4 dimethicone, PEG/PPG-15/15 dimethicone, PEG/PPG-16/2 dimethicone, PEG/PPG-17/18 dimethicone, PEG/PPG-18/18 dimethicone, PEG/PPG-19/19 dimethicone, PEG/PPG-20/6 dimethicone, PEG/PPG-20/15 dimethicone, PEG/PPG-20/20 Dimethicone, PEG/PPG-20/23 dimethicone, PEG/PPG-20/29 dimethicone, PEG/PPG-22/23 dimethicone, PEG/PPG-22/24 dimethicone, PEG/PPG-23/6 dimethicone, PEG/PPG-25/25 dimethicone, PEG/PPG-27/27, bis-PEG/PPG 18/6 dimethicone, bis-PEG/PPG-20/20 dimethicone, PEG/PPG-18/18 dimethicone, cetyl PEG/PPG-10/1 dimethicone, lauryl PEG/PPG-18/18 methicone, PEG/PPG 20/22 butyl ether dimethicone, PEG/PPG 23/6 dimethicone, PEG/PPG 20/15 dimethicone, PEG/PPG 20/23 dimethicone, or a combination thereof;
(b) about 2 to about 20 wt. %, preferably about 3 to about 15 wt. %, more preferably about 5 to about 12 wt. % of propylene glycol;
(c) about 1 to about 12 wt. %, preferably about 2 to about 10 wt. %, more preferably about 2 to about 6 wt. % of one or more cationic surfactants selected from:
   more quaternary ammonium compounds selected from cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, and mixtures thereof; and/or
   one or more fatty dialkylamines selected from oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, salts thereof, or a combination thereof;
(d) about 1 to about 15 wt. %, preferably about 2 to about 12 wt. %, more preferably about 5 to about 10 wt. % of one or more fatty alcohols selected from decyl alcohol, undecyl alcohol, dodecyl alcohol, myristyl alcohol, cetyl alcohol, stearyl alcohol, isostearyl alcohol, isocetyl alcohol, behenyl alcohol, linalool, oleyl alcohol, myricyl alcohol, or a mixture thereof;
(e) about 55 to about 85 wt. %, preferably about 60 to about 85 wt. %, more preferably about 60 to about 80 wt. % of water;
(f) about 0.01 to about 6 wt. %, preferably about 0.1 to about 5 wt. %, more preferably about 0.5 to about 4 wt. % of one or more nonionic thickening polymers selected from polysaccharides and associative polymers, in particular, sclerotium gum, guar gums, hydroxyalkyl celluloses optionally modified with a hydrophobic group, such as hydroxyethylcelluloses, hydroxymethylcelluloses optionally modified with a hydrophobic group, and inulins optionally modified with a hydrophobic group;
(g) about 0.1 to about 10 wt. %, preferably about 0.5 to about 8, more preferably about 1 to about 6 wt. % of one or more alkyl betaines selected from cocobetaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, and mixtures thereof. Typically, at least one betaine compound is selected from coco betaine, cocamidopropyl betaine, behenyl betaine, capryl/capramidopropyl betaine, and lauryl betaine, and mixtures thereof, more preferably cocobetaine;
(h) optionally, about 0.1 to about 10 wt. %, preferably about 0.1 to about 8 wt. %, more preferably about 0.5 to about 6 wt. % of one or more amino-functionalized silicone oils, preferably amodimethicone;
(i) optionally, about 0.01 to about 10 wt. %, preferably about 0.1 to about 8 wt. %, more preferably about 0.1 to about 5 wt. % of one or more water-soluble organic solvents, preferably wherein the one or more water-soluble organic solvents are selected from mono-alcohols (for example C₁₋₆ or C₁₋₄ mono-alcohols), polyols (polyhydric alcohols), glycols other than the glycols of (b), and a mixture thereof, more preferably selected from C₁₋₆ or C₁₋₄ mono-alcohols;
(j) about 0.01 to about 10 wt. %, preferably about 0.1 to about 8 wt. %, more preferably about 1 to about 5 wt. % of one or more miscellaneous ingredients, preferably one or more miscellaneous ingredients selected from preservatives, fragrances, pH adjusters, salts, chelating agents, buffers, antioxidants, flavonoids, vitamins, amino acids (e.g., taurine), botanical extracts, UV filtering agents, proteins, protein hydrolysates, and/or isolates, fillers (e.g., organic and/or inorganic fillers such as talc, calcium carbonate, silica, particular materials, etc.) composition colorants, or a mixture thereof;
   wherein all weight percentages are based on a total weight of the composition.

### Optional Features

In various embodiments, the hair treatment compositions described throughout the instant disclosure are free or essentially free from anionic surfactants. In other embodiments, the hair treatment composition includes less than 1 wt. % of anionic surfactants. In further embodiments, the hair treatment composition includes less than 0.8 wt. %, less than 0.5 wt. %, less than 0.1 wt. %, or less than 0.05 wt. % of anionic surfactants. In other embodiments, the hair treatment compositions optionally include up to about 2, 3, or 5 wt. % of one or more anionic surfactants.

In various embodiments, the hair treatment compositions of the instant disclosure are free or essentially free from glycerin (glycerol). In other embodiments, the hair treatment composition includes less than 1 wt. % of glycerin. In further embodiments, the hair treatment composition includes less than 0.8 wt. %, less than 0.5 wt. %, less than 0.1 wt. %, or less than 0.05 wt. % of glycerin. In other embodiments, the hair treatment compositions optionally include up to about 2, 3, or 5 wt. % of glycerin.

In various embodiments, the hair treatment compositions of the instant disclosure are free or essentially free from silicones other than the (a) one or more polyether-modified polysiloxanes and the (h) one or more amino-functionalized silicones. For example, the compositions may be free or essentially free from dimethicone, methicone, dimethiconol, and the like. In other embodiments, the hair treatment composition includes less than 1 wt. % of silicones other than the (a) one or more polyether-modified polysiloxanes and the (h) one or more amino-functionalized silicones. In further embodiments, the hair treatment composition includes less than 0.8 wt. %, less than 0.5 wt. %, less than 0.1 wt. %, or less than 0.05 wt. % of silicones other than the (a) one or more polyether-modified polysiloxanes and the (h) one or more amino-functionalized silicones. In other embodiments, the hair treatment compositions optionally include up to about 2, 3, or 5 wt. % of one or more silicones other than the (a) one or more polyether-modified polysiloxanes and the (h) one or more amino-functionalized silicones.

In various embodiments, the hair treatment compositions of the instant disclosure are free or essentially free from hair styling polymers (film-forming polymers) other than cationic conditioning polymers of (c), which may optionally exhibit film forming properties. Non-limiting examples of hair styling polymers (or film forming polymers) include nonionic hair styling polymers (or nonionic film forming polymers), anionic hair styling polymers (or anionic film forming polymers), amphoteric hair styling polymers (or amphoteric film forming polymers), or a combination thereof. Cationic styling polymers (cationic film forming polymers) may also be excluded or essentially excluded to the extent such cationic hair styling polymers (or cationic film forming polymers) are not included as a cationic conditioning agent of (c). As mentioned elsewhere throughout the disclosure the one or more cationic conditioning agents of (c) encompass cationic surfactants and cationic conditioning polymers. Some cationic conditioning polymers have film-forming properties and some cationic film-forming polymers have conditioning properties. Accordingly, the hair treatment compositions of the instant disclosure can optionally include one or more cationic conditioning polymers even if the cationic conditioning polymers have film-forming qualities. Alternatively, the hair treatment compositions of the instant disclosure can optionally include one or more cationic conditioning polymers that do not necessarily provide appreciable film-forming properties to the hair (but provide conditioning properties to the hair) and therefore it is conceivable that such hair styling compositions could be free or essentially free from cationic film-forming polymers.

Nonlimiting examples of nonionic hair styling polymers (nonionic film forming polymers) include PVPs (polyvinylpyrrolidones) and N-vinylpyrrolidone/vinylacetate copolymers; and polymers obtained from polymerization of at least one type of monomers selected from vinylpyrrolidone, vinylcaprolactam, vinyl esters, vinyl alcohol, vinyl acetate, (meth)acrylamide, and/or its derivatives, (meth)acrylic acid, propylene and/or ethylene glycol acid. crotonic acid, or mixtures thereof.

Nonlimiting examples of amphoteric hair styling polymers (amphoteric film forming polymers) include octylacrylamide/acrylate/butylaminoethyl methacrylate copolymers. alkylacrylamide/alkylaminoalkyl methacrylate/(meth)acrylic acid copolymers, copolymers which are formed from at least one first monomer type which has quaternary amine groups, and at least one second monomer type which has acid groups, copolymers of fatty alcohol acrylates, of alkylamine oxide methacrylate and at least one monomer chosen from acrylic acid and methacrylic acid, methacryloylethylbetaine/methacrylic acid and/or esters copolymers, polymers preparable from quaternary croton betaines or quaternary croton betaine esters, or mixtures thereof, or a mixture thereof.

Nonlimiting examples of anionic hair styling polymers (anionic film-forming polymers) include copolymers of vinyl acetate and crotonic acid; terpolymers of vinyl acetate, crotonic acid and a vinyl ester of an alpha-branched saturated aliphatic monocarboxylic acid such as vinyl neo-decanoate; copolymers of methyl vinyl ether and maleic anhydride wherein such copolymers are esterified with a saturated alcohol containing from 1 to 4 carbon atoms such as ethanol or butanol; acrylic copolymers containing acrylic acid or methacrylic acid as the anionic radical-containing moiety with other monomers such as: esters of acrylic or methacrylic acid with one or more saturated alcohols having from 1 to 22 carbon atoms (such as methyl methacrylate, ethyl acrylate, ethyl methacrylate, n-butyl acrylate, t-butyl acrylate, t-butyl methacrylate, n-butyl methacrylate, n-hexyl acrylate, n-octyl acrylate, lauryl methacrylate and behenyl acrylate); glycols having from 1 to 6 carbon atoms (such as hydroxypropyl methacrylate and hydroxyethyl acrylate); styrene; vinyl caprolactam; vinyl acetate; acrylamide; alkyl acrylamides and methacrylamides having 1 to 8 carbon atoms in the alkyl group (such as methacrylamide, t-butyl acrylamide and n-octyl acrylamide); and other compatible unsaturated monomers.

Further nonlimiting examples of anionic hair styling polymers (anionic film forming polymers) include carboxylated polyurethanes. Carboxylated polyurethane resins are linear, hydroxyl-terminated copolymers having pendant carboxyl groups. They may be ethoxylated and/or propoxylated at least at one terminal end. The carboxyl group can be a carboxylic acid group or an ester group, wherein the alkyl moiety of the ester group contains one to three carbon atoms. The carboxylated polyurethane resin can also be a copolymer of polyvinylpyrrolidone and a polyurethane, having a CTFA designation PVP/polycarbamyl polyglycol ester. Further nonlimiting examples include anionic (meth)acrylate copolymers, particularly copolymers of at least two monomers selected from the group consisting of acrylic acid, methacrylic acid, C.sub.1-6-alkylester of acrylic acid, C1-6-alkylester of methacrylic acid, C2-10-aminoalkylester of acrylic acid, C2-10-aminoalkylester of methacrylic acid, C1-6-alkylamides of acrylic acid and C1-6-alkylamides of methacrylic acid.

In various embodiments, the hair treatment compositions of the instant disclosure are free or essentially free from hair styling polymers (film forming polymers) other than cationic conditioning polymers of (c), which may optionally exhibit film forming properties. In further embodiments, the hair treatment composition includes less than 1 wt. % of hair styling polymers (film forming polymers) other than cationic conditioning polymers of (c), which may optionally exhibit film forming properties. In further embodiments, the hair treatment composition includes less than 0.8 wt. %, less than 0.5 wt. %, less than 0.1 wt. %, or less than 0.05 wt. % of hair styling polymers (film forming polymers). In other embodiments, the hair treatment compositions optionally include up to about 2, 3, or 5 wt. % of hair styling polymers (film forming polymers) other than cationic conditioning polymers of (c), which may optionally exhibit film forming properties.

In various embodiments, the hair treatment compositions of the instant disclosure are free or essentially free from hair anionic hair styling polymers (anionic film forming polymers). In further embodiments, the hair treatment composition includes less than 1 wt. % of anionic hair styling polymers (anionic film forming polymers). In further embodiments, the hair treatment composition includes less than 0.8 wt. %, less than 0.5 wt. %, less than 0.1 wt. %, or less than 0.05 wt. % of anionic hair styling polymers (anionic film forming polymers). In other embodiments, the hair treatment compositions optionally include up to about 2, 3, or 5 wt. % of anionic hair styling polymers (anionic film forming polymers).

In various embodiments, the hair treatment compositions of the instant disclosure are free or essentially free from amphoteric hair styling polymers (amphoteric film forming polymers). In further embodiments, the hair treatment composition includes less than 1 wt. % of amphoteric hair styling polymers (amphoteric film forming polymers). In further embodiments, the hair treatment composition includes less than 0.8 wt. %, less than 0.5 wt. %, less than 0.1 wt. %, or less than 0.05 wt. % of amphoteric hair styling polymers (amphoteric film forming polymers). In other embodiments, the hair treatment compositions optionally include up to about 2, 3, or 5 wt. % of amphoteric hair styling polymers (amphoteric film forming polymers).

In various embodiments, the hair treatment compositions of the instant disclosure are free or essentially free from non-silicone-based oils (e.g., mineral oil, alkanes (paraffins), plant oils, and the like other than the fatty alcohols of (d). In further embodiments, the hair treatment composition includes less than 1 wt. % of non-silicone-based oils other than the fatty alcohols of (d). In further embodiments, the hair treatment composition includes less than 0.8 wt. %, less than 0.5 wt. %, less than 0.1 wt. %, or less than 0.05 wt. % of non-silicone-based oils other than the fatty acids of (d). In other embodiments, the hair treatment compositions optionally include up to about 2, 3, or 5 wt. % of one or more non-silicone-based oils in addition to the fatty alcohols of (d).

In various embodiments, the hair treatment compositions of the instant disclosure are free or essentially free from waxes (fatty compounds that are solid at room temperature) except for the fatty alcohols of (d). In further embodiments, the hair treatment composition includes less than 1 wt. % of waxes (fatty compounds that are solid at room temperature) in addition to the fatty alcohols of (d). In further embodiments, the hair treatment composition includes less than 0.8 wt. %, less than 0.5 wt. %, less than 0.1 wt. %, or less than 0.05 wt. % of waxes (fatty compounds that are solid at room temperature) other than the fatty alcohols of (d). In other embodiments, the hair treatment compositions optionally include up to about 2, 3, or 5 wt. % of one or more waxes (fatty compounds that are solid at room temperature) in addition to the fatty alcohols of (d).

pH

The pH of the hair treatment compositions can vary. Nonetheless, in various embodiments, the pH of the hair treatment compositions is from about 4 to about 10. IN further embodiments, the pH of the hair treatment composition is from about 4 to about 9, about 4 to about 8, about 4 to about 7, about 5 to about 10, about 5 to about 9, about 5 to about 8, about 5 to about 7, about 6 to about 10, about 6 to about 9, about 6 to about 8, or about 6 to about 7. In other embodiments, the pH of the hair treatment composition is less than 9, less than 8, or less than 8, wherein the minimum pH is about 4, 5, or 6.

### Viscosity

The viscosity of the hair treatment compositions will vary. Nonetheless, in various embodiments, the viscosity of the hair treatment compositions is about 100 to about 100,000 cPs, measured with a Brookfield DV-LL+Pro Viscometer using Spindle T bar-C and rotational speed of 10% RPM at 25ºC. In further embodiments, the viscosity is from about 500 to about 100,000 cPs, about 1,000 to about 100,000 cps, about 5,000 to about 100,000 cPs, about 100 to about 80,000 cps, about 500 to about 80,000 cps, about 1,000 to about 80,000 cps, about 5,000 to about 80,000 cps, about 100 to about 60,000 cPs, about 500 to about 60,000 cPs, about 1,000 to about 60,000 cPs, about 5,000 to about 60,000 cPs, about 100 to about 50,000 cPs, about 500 to about 50,000 cPs, about 1,000 to about 50,000 cPs, about 5,000 to about 50,000 cPs, about 100 to about 25,000 cPs, about 500 to about 25,000 cPs, about 1,000 to about 25,000 cPs, about 5,000 to about 25,000 cPs, about 100 to about 15,000 cPs, about 500 to about 15,000 cps, about 1,000 to about 15,000 cPs, or about 5,000 to about 15,000 cPs, measured with a Brookfield DV-LL+Pro Viscometer using Spindle T bar-C and rotational speed of 10% RPM at 25ºC.

### Forms

The hair treatment compositions are typically oil-in-water emulsions. Alternatively, the compositions can be oil dispersions. The compositions typically have a translucent or opaque appearance and can be in the form of a cream, gel, lotion, rinse, and the like. In various embodiments, the hair treatment compositions are not an aerosol. In further embodiments, the hair treatment compositions are not a foam. In further embodiments, the hair treatment compositions are preferably oil-in-water emulsions that are translucent or opaque with the consistency of a cream, a gel, or a lotion.

The compositions are unique because they are effective as leave-on products and effective as rinse-off products. The compositions interact with the hair fibers with a type of “stickiness” (attraction) such that the shine enhancing properties withstand rinsing. In this respect, the compositions can be used like a traditional conditioner composition applied after shampooing the hair. Moreover, the compositions can be used even after conditioning to provide additional shine and luster to the hair, regardless of whether the compositions are used as a leave-on or rinse-off product.

### Kits

The hair treatment compositions of the instant disclosure may be included in a kit. For example, a kit comprising one or more hair-treatment compositions according to the instant disclosure and one or more additional hair treatment compositions, wherein the hair treatment composition(s) of the instant disclosure and the additional hair treatment composition(s) are separately contained. The type and purpose of the one or more additional hair treatment compositions are not limited. Nonetheless, in various embodiments, the one or more additional hair treatment compositions are selected from shampoo compositions, conditioning compositions, styling compositions, finishing compositions, or a combination thereof. In further embodiments, a hair treatment composition of the instant disclosure is included in a kit with a shampoo composition, wherein the hair treatment composition of the instant disclosure and the shampoo composition are separately contained.

### Methods

The hair treatment compositions may be used in various methods for improving the look and feel of hair, for example, human hair, including human hair an individual's head. For example, the hair treatment compositions are useful for: (i) imparting shine and luster to hair; (ii) improving texture and feel of hair; (iii) preventing or reducing frizz, especially in high humidity conditions; and (iv) improving the appearance of hair; wherein the methods typically comprise applying a hair treatment composition disclosed herein to the hair. The hair treatment composition can be applied to the hair as a leave-on product (i.e., allowed to remain on the hair without rinsing and the hair subsequently styled) or as a rinse-out product (i.e., rinsed from the hair prior to subsequently styling the hair).

When used as a rinse-out product, the hair treatment composition is typically applied to wet or dry hair, allowed to remain on the hair for a period of time, and subsequently rinsed from the hair prior to styling the hair. For example, the hair-treatment composition may be allowed to remain on the hair for about 10 seconds to about 15 minutes. Furthermore, the hair-treatment composition can remain on the hair for about 10 seconds to about 10 minutes, about 10 seconds to about 5 minutes, about 10 seconds to about 3 minutes, about 30 seconds to about 15 minutes, about 30 seconds to about 10 minutes, about 30 seconds to about 5 minutes. More simply, when used as a rinse-off product, the hair treatment composition can be used like a traditional conditioner. However, unlike a traditional conditioner, the hair treatment compositions are not necessarily used in conjunction with a shampooing step. In some instances, the hair is initially cleansed with a shampoo and subsequently treated with a hair treatment composition according to the instant disclosure. In other instances, the hair treatment composition is used as a stand-alone product such that it is not necessary for use in conjunction with cleansing the hair with a shampoo. Furthermore, the hair treatment compositions of the instant disclosure can be used even after the hair has been treated with a conditioner, for example, to provide additional shine and luster to the hair.

When used as a leave-on product, the hair treatment composition can be applied to wet, damp, or dry hair. As the term suggests, a leave-on product remains on the hair for an extended period of time without being rinsed from the hair. The hair treatment composition is applied to the hair and the hair is subsequently styled and allowed to dry without rinsing the hair treatment composition from the hair. In various embodiments, after application of the hair treatment composition, the hair can be dried, for example, with a blow drying. In further embodiments, the hair can be treated with a hot iron, for example, a flat iron or a curling iron.

Implementation of the present disclosure is provided by way of the following examples. The examples serve to illustrate the technology without being limiting in nature.

### Example 1

### Compositions

| | | | Inventive | | | | | Comparative | |
|---|---|---|---|---|---|---|---|---|---|
| | | | A | B | C | D | E | F | G |
| (a) | Polyether- | PEG-12 DIMETHICONE | | | | 1.5 | | | |
| | Modified | PEG/PPG-4/12 | 1.5 | 1.5 | 1.5 | | | 1.5 | 1.5 |
| | | DIMETHICONE | | | | | | | |
| | Polysiloxanes | PEG-14 DIMETHICONE | | | | | 1.4 | | |
| (b) | Glycol | PROPYLENE GLYCOL | 7 | 7 | 7 | 7 | 7 | 7 | |
| | | GLYCERIN | | | | | | | 7 |
| (c) | Cationic | BEHENTRIMONIUM | 3.2 | | | 3.2 | 3.2 | | 3.2 |
| | Surfactants | CHLORIDE | | | | | | | |
| | | CETRIMONIUM | 0.02 | 0.02 | 3.2 | 0.02 | 0.02 | | 0.02 |
| | | CHLORIDE | | | | | | | |
| | | STEARAMIDOPROPYL | | 3.2 | | | | | |
| | | DIMETHYLAMINE | | | | | | | |
| (d) | Fatty Alcohol | CETEARYL ALCOHOL | 7 | 7 | 7 | 7 | 7 | 7 | 7 |
| (f) | Nonionic | SCLEROTIUM GUM | 1 | 1 | 1 | 1 | 1 | 1 | 1 |
| | Thickening | | | | | | | | |
| | Polymer | | | | | | | | |
| (g) | Amphoteric | COCAMIDOPROPYL | 3.8 | 3.8 | 3.8 | 3.8 | 3.8 | 3.8 | 3.8 |
| | Surfactant | BETAINE | | | | | | | |
| | Nonionic | TRIDECETH-6 | 0.1 | 0.1 | 0.1 | 0.1 | 0.1 | 0.1 | 0.1 |
| | Surfactant | | | | | | | | |
| (h) | Amino Silicone | AMODIMETHICONE | 1 | 1 | 1 | 1 | 1 | 1 | 1 |
| (i) | Water-Soluble | ISOPROPYL ALCOHOL | 0.7 | | | 0.7 | 0.7 | 0.07 | 0.07 |
| | Solvent | | | | | | | | |
| (j) | Miscellaneous | fragrances, | ≤5 | ≤5 | ≤5 | ≤5 | ≤5 | ≤5 | ≤5 |
| | Ingredients | preservatives, chelating | | | | | | | |
| | | agents, alkanolamines, | | | | | | | |
| | | salts, pH adjusters, etc. ¹ | | | | | | | |
| (e) | Water | WATER | qs | qs | qs | qs | qs | qs | qs |

| pH | 6.7 | 6.7 | 6.7 | 6.7 | 6.7 | 6.7 | 6.7 |
|---|---|---|---|---|---|---|---|
| Viscosity | Between 10,000 to 12,000 cPs* | | | | | | |
| Shine | +++ | +++ | +++ | +++ | +++ | + | + |

¹ Ethanolamine, sodium chloride, citric acid, fragrance pentaerythrityl tetra-di-t-buyl hydroxyhydrocinnamate, phenoxyethanol, chlorhexidine digluconate.
² Viscosity measure at 25° C. with a Brookfield DV-LL + Pro Viscometer using Spindle T bar-C and rotational speed of 10% RPM.

### Example 2

### Shine Testing

The compositions set forth in Example 1 were tested to determine their ability to impart shine to hair. Caucasian virgin hair swatches (3 gram, 25-inch) were cleansed with a standard shampoo and excess water removed after cleansing. The hair swatches were separately treated with one of the compositions set forth in Example 1. Each of the compositions was applied to damp hair swatches, messaged into the hair swatches, and allowed to remain on the hair swatches for 3 minutes. After 3 minutes, the swatches were rinsed with water and blow dried. Two expert evaluators then independently assessed shine of the hair swatches based on the following scale:
+++=Very High Shine
++=High Shine
+=Slight Shine.

The results are shown in the table of Example 1 but reproduced below.

| Compositions | A | B | C | D | E | F | G |
|---|---|---|---|---|---|---|---|
| Shine | +++ | +++ | +++ | +++ | +++ | + | + |

The results show that a combination of polyether-modified polysiloxanes, propylene glycol, and cationic surfactant in the compositions imparted a substantial increase in shine to the hair. This is illustrated by the results for Comparative Composition F, which lacked cationic surfactant. The data also show that propylene glycol, in combination with the polyether-modified polysiloxanes and cationic surfactant contributes to the surprising improvement of shine. This is illustrated by the results for Comparative Composition G. Comparative Composition G included glycerin instead of propylene glycol. This is surprising considering that glycerin and propylene glycol often provide similar properties to cosmetic compositions. The compounds are structurally very similar, differing by one hydroxy group.

### Example 3

### Compositions and Shine Assessment

| | | | Inven- | Compar- |
|---|---|---|---|---|
| | | | tive | ative |
| | | | H | I |
| (a) | Polyether-Modified | PEG/PPG-4/12 | 1.5 | |
| | Polysiloxanes | DIMETHICONE | | |
| (b) | Glycol | PROPYLENE GLYCOL | 7 | 2 |
| (c) | Cationic Surfactant | BEHENTRIMONIUM | 4 | 4 |
| | | CHLORIDE | | |
| | | CETRIMONIUM | 0.02 | 0.02 |
| | | CHLORIDE | | |
| (d) | Fatty Alcohol | CETEARYL ALCOHOL | 7 | 7 |
| (f) | Nonionic | SCLEROTIUM GUM | 1 | 1 |
| | Thickening Agent | | | |
| (g) | Amphoteric | COCAMIDOPROPYL | 3.8 | 3.8 |
| | Surfactant | BETAINE | | |
| | Nonionic Surfactant | TRIDECETH-6 | 0.1 | 0.1 |
| (h) | Amino Silicone | AMODIMETHICONE | 1.0 | 1.0 |
| (j) | Miscellaneous | MISCELLANEOUS | ≤5 | ≤5 |
| | Ingredients | | | |
| (e) | Water | WATER | QS | QS |

Inventive Composition H and Comparative Composition I were tested to determine their ability to impart shine to hair. Caucasian virgin hair swatches (3 gram, 25-inch) were cleansed with a standard shampoo and excess water removed after cleansing. The hair swatches were separately treated with Inventive Composition H or Comparative Composition I. Each of the compositions was applied to damp hair swatches, messaged into the hair swatches, and allowed to remain on the hair swatches for 3 minutes. After 3 minutes, the swatches were rinsed with water and blow dried.

After drying the hair swatches were dried, they were evaluated using a SAMBA SHINER Instrument (by Bossa Nova Vision), which is a device commonly used in the cosmetics industry to measure hair shine (also called hair luster or hair gloss). It provides quantitative measurement to substantiate claims and evaluate products efficacy. Results from the SAMBA SHINE® instrument showed considerably better shine for the hair treated with Inventive Composition H over the hair treated with Comparative Composition I. For hair treated with Inventive Composition H, the SAMBA SHINE® Instrument showed nearly a doubling in the range of shine area (a measurement representing an extent of shine). In addition, the results showed the shine of the hair treated with Inventive Compositions was visibly brighter (more reflective) than the shine provided by Comparative Composition H. In other words, the shine provided by Inventive Composition H was appreciably more pronounced and brighter (more reflective) than shine provided by Comparative Composition I.

The foregoing description illustrates and describes the disclosure. Additionally, the disclosure shows and describes only the preferred embodiments but as mentioned above, it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be include, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art.

The expression “one or more” means “at least one” and thus includes individual components as well as mixtures/combinations.

The term “plurality” means “more than one” or “two or more.”

Other than in the operating examples, or where otherwise indicated, all numbers expressing quantities of ingredients and/or reaction conditions may be modified in all instances by the term “about,” meaning within +/−5% of the indicated number.

All percentages, parts and ratios herein are based upon the total weight of the compositions, unless otherwise indicated.

Some of the various categories of components identified may overlap. In such cases where overlap may exist and the composition includes both components (or the composition includes more than two components that overlap), an overlapping compound does not represent more than one component. For example, certain compounds may be considered both an surfactant and a fatty compound. If a particular composition includes both a surfactant and a fatty compound, a single compound will serve as only the surfactant or the fatty compound (the single compound does not serve as both the surfactant and the fatty component).

A “rinse-off” product refers to a composition that is rinsed and/or washed from the hair with water either after or during the application of the composition onto the hair, and before drying and/or styling the hair. At least a portion of the composition is removed from the hair during the rinsing and/or washing.

A “leave-on” product refers to a composition that is not rinsed and/or washed from the hair after or during application of the composition onto the hair. The composition remains on the hair during drying and/or throughout styling.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The hair treatment compositions of the instant disclosure may include one or more surfactants and/or emulsifiers, for example, one or more nonionic, anionic, cationic, and/or amphoteric/zwitterionic surfactants or emulsifiers. The terms “surfactants” and “emulsifiers” include salts of the surfactants and emulsifiers even if not explicitly stated. In other words, whenever the disclosure refers to a surfactant or emulsifier, it is intended that salts are also encompassed to the extent such salts exist, even though the specification may not specifically refer to a salt (or may not refer to a salt in every instance throughout the disclosure), for example, by using language such as “a salt thereof” or “salts thereof.” Sodium and potassium are common cations that form salts with surfactants and emulsifiers. However, additional cations such as ammonium ions, or alkanolammonium ions such as monoethanolammonium or triethanolammonium ions, may also form salts of surfactants.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, less than about 0.01 wt. % or none of the specified material.

All components that are positively set forth in the instant disclosure may be negatively excluded from the claims, e.g., a claimed composition may be “free” or “essentially free” (or “substantially free”) of one or more components that are positively set forth in the instant disclosure. As an example, the hair treatment composition of the instant disclosure may optionally include one or more nonionic film-forming polymers (or film-setting or hair-styling polymers). However, the hair treatment compositions may be free or essentially free from the one or more nonionic film-forming polymers (or film-setting or hair-styling polymers).

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A hair treatment composition comprising:
(a) about 1 to about 10 wt. % of one or more polyether-modified polysiloxanes;
(b) about 2 to about 20 wt. % of one or more glycols selected from ethylene glycol, propylene glycol, butylene glycol, pentylene glycol, dipropylene glycol, or a combination thereof;
(c) about 1 to about 12 wt. % of one or more cationic conditioning agents;
(d) about 1 to about 15 wt. % of one or more fatty alcohols; and
(e) water;
wherein the composition is an oil-in-water emulsion, and all weight percentages are based on a total weight of the composition.

2. The composition of claim 1, wherein at least one of the one or more glycols is propylene glycol.

3. The composition of claim 1, wherein the one or more polyether-modified polysiloxanes are selected from PEGylated dimethicones, bis-PEGylated dimethicones, PEG/PPG dimethicones, or a combination thereof.

4. The composition of claim 3, wherein:
the PEGylated dimethicones are selected from PEG-10 dimethicone, PEG-9 dimethicone, PEG-8 dimethicone, PEG-3 dimethicone, PEG-7 dimethicone, PEG-12 dimethicone, PEG-11 methyl ether dimethicone, PEG-32-dimethicone, PEG-14 dimethicone, PEG-17 dimethicone, or a combination thereof;
the bis-PEGylated dimethicones are selected from bis-PEG-[10-20] dimethicone, bis-PEG-10 dimethicone, bis-PEG-12 dimethicone, bis-PEG-17 dimethicone, bis-PEG-20 dimethicone, or a combination thereof; and
the PEG/PPG dimethicones are selected from PEG/PPG-3/10 dimethicone, PEG/PPG-4/12 dimethicone, PEG/PPG-6/11 dimethicone, PEG/PPG-8/14 dimethicone, PEG/PPG-14/4 dimethicone, PEG/PPG-15/15 dimethicone, PEG/PPG-16/2 dimethicone, PEG/PPG-17/18 dimethicone, PEG/PPG-18/18 dimethicone, PEG/PPG-19/19 dimethicone, PEG/PPG-20/6 dimethicone, PEG/PPG-20/15 dimethicone, PEG/PPG-20/20 Dimethicone, PEG/PPG-20/23 dimethicone, PEG/PPG-20/29 dimethicone, PEG/PPG-22/23 dimethicone, PEG/PPG-22/24 dimethicone, PEG/PPG-23/6 dimethicone, PEG/PPG-25/25 dimethicone, PEG/PPG-27/27, bis-PEG/PPG 18/6 dimethicone, bis-PEG/PPG-20/20 dimethicone, PEG/PPG-18/18 dimethicone, cetyl PEG/PPG-10/1 dimethicone, lauryl PEG/PPG-18/18 methicone, PEG/PPG 20/22 butyl ether dimethicone, PEG/PPG 23/6 dimethicone, PEG/PPG 20/15 dimethicone, PEG/PPG 20/23 dimethicone, or a combination thereof.

5. The composition of claim 1, wherein the one or more cationic conditioning agents are selected from cationic surfactants, cationic polymers, or a combination thereof.

6. The composition of claim 5 comprising one or more cationic surfactants.

7. The composition of claim 5, wherein the one or more cationic surfactants are selected from quaternary ammonium compounds, fatty dialkylamines, fatty amidoamines, salts thereof, or a combination thereof.

8. The composition of claim 7 comprising one or more quaternary ammonium compounds selected from cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, and mixtures thereof.

9. The composition of claim 7 comprising one or more fatty dialkylamines selected from oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, salts thereof, or a combination thereof.

10. The composition of claim 1, wherein the one or more fatty alcohols are selected from decyl alcohol, undecyl alcohol, dodecyl alcohol, myristyl alcohol, cetyl alcohol, stearyl alcohol, isostearyl alcohol, isocetyl alcohol, behenyl alcohol, linalool, oleyl alcohol, myricyl alcohol, or a mixture thereof.

11. The composition of claim 1 comprising about 55 to about 90 wt. % of water.

12. The composition of claim 1, further comprising:
(f) one or more nonionic thickening polymers.

13. The composition of claim 12, wherein the one or more nonionic thickening polymers are selected from polysaccharides, polysaccharide derivatives, or a combination thereof.

14. The composition of claim 13, wherein the one or more nonionic thickening polymers are selected from sclerotium gum, guar gums, guar gum derivatives, xanthan gum, hydroxyalkyl celluloses optionally modified with a hydrophobic group, inulins, carrageenans, carrageenan derivatives, or a combination thereof.

15. The composition of claim 1, further comprising:
(g) one or more surfactants other than cationic surfactants.

16. The composition of claim 15 comprising one or more alkyl betaines.

17. The composition of claim 1, further comprising:
(h) one or more amino-functionalized silicone oils.

18. A hair treatment composition comprising:
(a) about 1 to about 10 wt. % of one or more polyether-modified polysiloxanes;
(b) about 2 to about 20 wt. % of propylene glycol;
(c) about 1 to about 10 wt. % of one or more cationic conditioning agents;
(d) about 1 to about 12 wt. % of one or more fatty alcohols;
(e) water;
(f) one or more nonionic thickening polymers;
(g) one or more alkyl betaines;
(h) optionally, one or more amino-functionalized silicone oils;
(i) optionally, one or more water-soluble organic solvents; and
(i) optionally, up to 5 wt. % of one or more miscellaneous ingredients;
wherein the composition is an oil-in-water emulsion and all weight percentages are based on a total weight of the composition.

19. A method for treating hair comprising applying the composition of claim 1 to the hair and subsequently rinsing the composition from the hair within about 1 hour from application to the hair.

20. The method of claim 19, wherein treating the hair imparts shine to the hair.
